(19) United States
(12) Patent Application Publication
(10) Pub. No.: US 2013/0017180 A1
(43) Pub. Date: Jan. 17, 2013

(54) PLATELET RICH PLASMA FORMULATIONS

(71) Applicant: Allan Mishra, Menlo Park, CA (US)

(72) Inventor: Allan Mishra, Menlo Park, CA (US)

(21) Appl. No.: 13/636,321

(22) PCT Filed: Apr. 5, 2011
(86) PCT No.: PCT/US11/31277

§ 371 Date: Sep. 20, 2012

Related U.S. Application Data
(60) Provisional application No. 61/320,862, filed on Apr. 5, 2010.
(60) Provisional application No. 61/351,178, filed on Jun. 3, 2010.

(51) Int. Cl. A61K 35/14; C12Q 1/02; C12M 1/34; A61P 9/00

(52) U.S. Cl. 424/93.71; 424/93.72; 435/29; 435/287.1

(57) ABSTRACT

Compositions for platelet rich plasma (PRP) and neutrophil-depleted PRP are provided. Methods for treating ischemia damaged tissues by delivering a PRP composition, in some embodiments a neutrophil-depleted PRP composition to the damaged tissue are provided. In some variations, the compositions may be useful to treat ischemic heart disease and repair damaged cardiovascular tissue following acute myocardial infarction including congestive heart failure. In some variations, the compositions may be useful to reduce cardiac apoptosis after a heart attack.

### RELATED APPLICATIONS

This application claims to the benefit of U.S. Provisional Patent Application No. 61/320,862, filed Apr. 5, 2010, and to U.S. Provisional Patent Application No. 61/351,178, filed Jun. 3, 2010, the entirety of which are incorporated herein by reference.

### FIELD OF THE INVENTION

The present application relates to systems, methods and compositions for treatment of tissue damage caused by ischemia. Some embodiments relate to formulations of platelet rich plasma (PRP) comprising different levels of platelets and white blood cells relative to whole blood. Some embodiments relate to the treatment of ischemic damage to cardiovascular tissue including any tissue with ischemia (low blood flow) by administration of PRP. Several embodiments relate to neutrophil depleted PRP compositions and methods of treating ischemic damage to cardiovascular tissue by administration of neutrophil depleted PRP.

### BACKGROUND

Ischemia is a restriction in the blood supply to tissue. The most common causes of ischemia are acute arterial thrombus (blood clot) formation, chronic narrowing (stenosis) of an artery, often caused by atherosclerotic disease, and arterial vasospasm. When tissue is deprived of blood supply for a period of time the tissue may be damaged both by the initial absence of oxygen (hypoxia) and nutrients, build up of metabolic waste products and by the return of the blood supply after the period of ischemia. If the hypoxic state is prolonged, cellular death may occur. Tissue may also be damaged by the returning blood supply after a period of ischemia, termed a reperfusion injury.

Highly metabolic organs, such as the heart, kidneys, and brain are the most quickly damaged by periods of ischemia, however, all bodily tissues require oxygen, glucose and other blood-borne factors and thus are susceptible to ischemic damage. Common types of ischemia include cardiac ischemia, ischemic colitis, mesenteric ischemia, brain ischemia (ischemic stroke), acute or chronic limb ischemia, and cutaneous ischemia.

Cardiac ischemia, also called myocardial ischemia, may be caused by ischemic heart disease, a chronic narrowing of the small blood vessels that supply blood and oxygen to the heart, or by heart attack, also known as acute myocardial infarction (MI), an acute blockage of the blood vessels supplying the heart. Cardiac ischemia damages the heart muscle, impairing the heart's ability to eject blood and therefore reduces ejection fraction. Ejection fraction is one of the most important predictors of prognosis; a significantly reduced ejection fraction typically is indicative of a poor prognosis. This reduction in the ejection fraction can manifest itself clinically as heart failure.

Ischemic heart disease is usually caused by a buildup of cholesterol and other substances, called plaque, in the arteries that bring oxygen to heart tissue and is the most common type of cardiomyopathy in the United States, affecting approximately 1 out of 100 people. Ischemic heart disease is a common cause of congestive heart failure. Ischemic heart disease may be treated with coronary artery bypass surgery or balloon angioplasty to improve blood flow to the damaged or weakened heart muscle. Other treatments of ischemic heart disease include the administration of ACE inhibitors, angiotensin receptor blockers (ARBs), diuretics, digitalis glycosides, beta-blockers and vasodilators. Ischemic heart disease commonly presents as an acute myocardial infarction.

Acute myocardial infarctions occur when the blood supply to the heart is interrupted, usually due to blockage of a coronary artery by an artherosclerotic plaque detaching from the artery wall. Acute myocardial infarction may comprise non-ST-elevated myocardial infarction or ST-elevated myocardial infarction. In an ST-elevated myocardial infarction, the ST segment in an electrocardiogram (ECG) is elevated, meaning that the ventricles do not depolarize as rapidly as they would in a healthy heart. If blood flow to the heart is impaired over an extended period of time, an ischemic cascade and cardiac apoptosis may occur, causing heart cells to die and not regenerate. In place of the ischemic tissue, scar tissue forms. The scar tissue may increase the likelihood of cardiac arrhythmia, and may result in the formation of ventricular aneurysms.

Reperfusion therapy is the standard of care for patients presenting with acute myocardial infarction and diagnostic testing suggestive of coronary artery occlusion. Reperfusion therapies include thrombolytic therapy, percutaneous coronary intervention (PCI), and/or bypass surgery. While reperfusion therapy restores blood flow to the ischemic tissue, it does not lessen the risk of arrhythmia resulting from the growth of scar tissue. A number of studies have demonstrated that rapid reperfusion therapy leads to significantly less myocardial injury and lower rates of heart failure and death. Despite efforts to achieve reperfusion quickly, significant myocardial injury and resulting heart failure and death still occur. Further, restoration of blood flow to the ischemic tissue may result in reperfusion injury where the restoration of circulation leads to inflammation and oxidative damage.

The death of the cardiomyocytes due to ischemic injury is followed by an inflammatory response as macrophages, monocytes, and neutrophils migrate into the infarct area upon return of blood flow. Expansion of the infarct site can then occur because of the activation of matrix metalloproteases, which degrade the extracellular matrix, resulting in weakening of the collagen scaffold, which results in wall thinning and ventricular dilation. Following this initial inflammatory phase, fibrillar, cross-linked collagen deposition, which resists deformation and rupture, is increased. This can result in negative left ventricular (LV) remodeling, leading to increased wall stress in the remaining viable myocardium and LV dilation. This remodeling may contribute to the progression of heart failure.

Despite improvements in drug therapies and interventional procedures, ischemic heart disease and myocardial infarction are leading causes of death in industrialized nations. Thus, there is a need for additional therapies to treat damaged myocardial tissue. Several new strategies have been developed to address this unmet clinical need including myocardial protection or preservation devices and biologic therapies. Among biologic therapies, adult stem cell therapy has shown promise based on studies demonstrating improved ejection fraction and/or decreased infarct size. Although stem cell-based therapies currently represent an exciting area of research, difficulty with cell availability, cell harvest morbidity, and timing of delivery has limited the adoption of these approaches. Importantly, the cost and potential risks of these approaches are also high and potentially prohibitive.

### SUMMARY

Whole blood can be fractionated into platelet rich plasma (PRP). PRP compositions may generally comprise platelet rich plasma that includes a specific concentration of platelets, red blood cells, and white blood cells. In some embodiments, the PRP compositions may be characterized relative to a baseline concentration of the platelets, red blood cells, and/or white blood cells of the whole blood from which the compositions are directly or indirectly derived. In some embodiments, the PRP compositions contain concentrated platelets and white blood cells which are higher than the baseline levels of the whole blood from which the PRP composition was derived. Several embodiments relate to formulations of PRP that contain an increased concentration of platelets and monocytes and or lymphocytes in comparison to neutrophils. Such formulations of PRP are referred to herein as granulocyte or neutrophil-depleted platelet rich plasma. In some embodiments, the neutrophil-depleted PRP composition is depleted in neutrophils at a level of 0 to 0.999 of the concentration of whole blood or complete elimination of the neutrophils from the composition.

In some embodiments, the PRP composition comprises platelet cells at a concentration at least 1.1 times the concentration of platelets in whole blood. The platelet concentration in the PRP composition may be between about 1.1 and about 2 times baseline, about 2 and about 4 times baseline, about 4 and about 6 times baseline, about 6 and about 8 times baseline, or higher. The platelet concentration in the PRP composition may be between about 500,000 and about 1,500,000 platelets per microliter.

The PRP composition may further comprise white blood cells (WBCs) at a higher concentration than white blood cells in whole blood. The WBC concentration may be between about 1.1 and about 2 times baseline, about 2 and about 4 times baseline, about 4 and about 6 times baseline, about 6 and about 8 times baseline, or higher. In some variations, the WBC concentration is about 15,000 to about 50,000 WBC per microliter.

In some embodiments, the PRP composition comprises specific concentrations of various types of white blood cells. The concentrations of lymphocytes and monocytes may be between about 1.1 and about 2 times baseline, about 2 and about 4 times baseline, about 4 and about 6 times baseline, about 6 and about 8 times baseline, or higher. The concentrations of eosinophils in the PRP composition may be about 1.5 times baseline. In some variations, the lymphocyte concentration is between about 5,000 and about 20,000 per microliter and the monocyte concentration is between about 1,000 and about 5,000 per microliter. The eosinophil may be between about 200 and about 1,000 per microliter.

In some embodiments, the PRP composition may contain a specific concentration of neutrophils. The neutrophil concentration may vary between less than the baseline concentration of neutrophils to eight times the baseline concentration of neutrophils. In some variations, the neutrophil concentration may be between 0 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. In preferred embodiments, the neutrophil concentration is less than the concentration in whole blood. In a more preferred embodiment, the neutrophil concentration is 0.1 to 0.9 the concentration found in whole blood, yet more preferably less than 0.1 the concentration found in whole blood. In a most preferred embodiment the neutrophils are eliminated or non-detectable in the PRP composition.

In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be 5 g/dl or less.

The PRP compositions disclosed herein may be useful in treating damaged connective tissue, cardiac tissue, and/or lung tissue. PRP compositions may also be useful in treating tissues with compromised blood flow, such as ischemic tissue in the legs, arms, brain or other organs. Specifically, acute or chronic limb ischemia may be treated with PRP compositions. In some embodiments, the PRP compositions described herein may repair tissue damage by slowing or halting apoptosis, and that the anti-apoptotic effects of the PRP compositions may be measured based on a decrease in caspases in the blood, such as caspase-3. In some embodiments, the PRP compositions may be applied in conjunction with reperfusion therapy. In some embodiments, the PRP compositions may further be applied with stem cell treatments.

In some embodiments, the PRP compositions disclosed herein may be useful in treating ischemia, cancer, a disease of the immune system, a connective tissue injury, a skin disease, or a disease of the nervous system. The composition may be useful for the treatment of acute or chronic skin conditions such as burns or wrinkles. The ischemia may be a brain ischemia or cardiac ischemia. The cancer may be brain cancer, thyroid cancer, pancreatic cancer, liver cancer, breast cancer, or prostate cancer. Other types of cancer or neoplasia may also be treated with this composition. The connective tissue injury may be a tendinosis, such as tennis elbow, rotator cuff injury, a knee injury, a spinal injury or plantar fasciitis. The nervous system disease may be Parkinsons' disease or other neurodegenerative disorders such as Alzheimers or Multiple Sclerosis.

Embodiments of the invention are directed to a method of treating ischemia damaged tissue comprising one or more of the following steps: obtaining a blood sample from the patient; obtaining PRP from the blood sample; obtaining an analysis of the cell-type composition of the PRP; performing an assay of potency as measured by an ELISA, genetic analysis (DNA, mRNA, miRNA or microarray) determining that the cell-type composition of the PRP indicates that it is suitable for treating the ischemia damaged tissue, and providing the PRP to the patient. In some embodiments, the ratio of monocytes and/or lymphocytes to neutrophils serves as an index to determine if the formulation may be efficaciously used as a treatment.

Embodiments of the invention are directed to a device having platelet rich plasma composition as described above alone or in combination with a fixation device such as a stent, suture, screw, or implantable device such as a patch. In some embodiments, the device is a chamber. In several embodiments, conditions in the chamber include one or more of the following: low oxygen tension, high oxygen tension, low pH, high pH, low pressure, high pressure, low UV, high UV, low temperature, and high temperature.

Several embodiments relate to a formulation of platelet rich plasma that contains 1.01 times baseline platelets or more in combination with 1.01 times baseline white blood cells or more. In some embodiments, the monocytes and/or lymphocytes are increased in comparison to neutrophils. In some embodiments, the neutrophils are depleted to 1% or more of baseline levels.

Several embodiments relate to the use of a formulation of platelet rich plasma containing 1.01 times baseline platelets or more in combination with 1.01 times baseline white blood cells or more to treat heart disease, lung disease, peripheral vascular disease, muscle, tendon, ligament, cartilage, bone, kidney, brain tissue and any other organ including pancreas, liver and skin. Some embodiments relate to the use of a formulation of platelet rich plasma containing 1.01 times baseline platelets or more in combination with 1.01 times baseline white blood cells or more wherein the monocytes and/or lymphocytes are increased in comparison to neutrophils to treat heart disease, lung disease, peripheral vascular disease, muscle, tendon, ligament, cartilage, bone, kidney, brain tissue and any other organ including pancreas, liver and skin. Some embodiments relate to the use of a formulation of platelet rich plasma containing 1.01 times baseline platelets or more in combination with 1.01 times baseline white blood cells or more wherein neutrophils are depleted to 1% or more of baseline levels to treat heart disease, lung disease, peripheral vascular disease, muscle, tendon, ligament, cartilage, bone, kidney, brain tissue and any other organ including pancreas, liver and skin.

Several embodiments relate to the use of a formulation of platelet rich plasma containing 1.01 times baseline platelets or more in combination with 1.01 times baseline white blood cells or more to treat a heart attack, congestive heart failure, chronic angina, critical limb ischemia, and/or any lung disease. Some embodiments relate to the use of a formulation of platelet rich plasma containing 1.01 times baseline platelets or more in combination with 1.01 times baseline white blood cells or more wherein the monocytes and/or lymphocytes are increased in comparison to neutrophils to treat a heart attack, congestive heart failure, chronic angina, critical limb ischemia, and/or any lung disease. Some embodiments relate to the use of a formulation of platelet rich plasma containing 1.01 times baseline platelets or more in combination with 1.01 times baseline white blood cells or more wherein neutrophils are depleted to 1% or more of baseline levels to treat a heart attack, congestive heart failure, chronic angina, critical limb ischemia, and/or any lung disease.

Some embodiments relate to a method of measuring the value of monocytes and/or lymphocytes to neutrophils which comprises measuring the ratio of monocytes and/or lymphocytes to neutrophils. In some embodiments, an increased ratio of monocytes and/or lymphocytes to neutrophils indicates that a PRP composition is suitable for use in treating heart attack, congestive heart failure, chronic angina, critical limb ischemia and/or any lung disease.

Several embodiments relate to a composition comprising platelets derived from whole blood at a first concentration of at least about 1.1 times a platelet concentration in the whole blood and white blood cells derived from the whole blood at a second concentration of at least a white blood cell concentration in the whole blood, wherein the white blood cells comprise neutrophils at a third concentration, wherein the third concentration is less than the neutrophil concentration in the whole blood and lymphocytes at a fourth concentration of at least 1.1 times a lymphocyte concentration in the whole blood; and monocytes at a fifth concentration of about 1.1 times a monocyte concentration in the whole blood. In some embodiments, the concentration of neutrophils is 1% of the concentration of neutrophils in whole blood. In several embodiments, the neutrophils are substantially eliminated from the composition. In some embodiments, the concentration of neutrophils is between about 2,000 neutrophils per microliter and about 3,000 neutrophils per microliter.

Several embodiments relate to a method of treating a cardiac condition comprising identifying cardiac ischemia in a patient and delivering a composition comprising platelets derived from whole blood at a first concentration of at least about 1.1 times a platelet concentration in the whole blood and white blood cells derived from the whole blood at a second concentration of at least a white blood cell concentration in the whole blood, wherein the white blood cells comprise neutrophils at a third concentration, wherein the third concentration is less than the neutrophil concentration in the whole blood and lymphocytes at a fourth concentration of at least 1.1 times a lymphocyte concentration in the whole blood; and monocytes at a fifth concentration of about 1.1 times a monocyte concentration in the whole blood to the patient to treat the cardiac condition. In some embodiments, the cardiac condition is ischemic heart disease or myocardial infarction. In some embodiments, the composition is prepared from the whole blood of the patient. In some embodiments, the composition prepared from the whole blood of the patient is tested prior to delivery to the patient. In several embodiments, the composition is delivered within about 24 hours of myocardial infarction. In some embodiments, the composition is delivered within about 24 hours of reperfusion therapy. Some embodiments relate to a method of treating a cardiac condition, wherein the composition further comprises one or more of fetal cardiomyocytes, embryonic stem cells, bone marrow cells, induced pluripotent stem cells, and cardiomyocytes derived from induced pluripotent stem cells and/or a biomaterial scaffold comprised of one or more of gelatin, alginate, collagen type 1 and Matrigel, polyglycolide, collagen, fibrin, or self-assembling peptides.

Several embodiments relate to a method of reducing apoptosis in ischemia damaged tissue, comprising delivering a PRP composition to a site of ischemic damage. Some embodiments relate to a method of reducing apoptosis in ischemia damaged tissue, comprising delivering a PRP composition comprising platelets derived from whole blood at a first concentration of at least about 1.1 times a platelet concentration in the whole blood and white blood cells at a second concentration of at least about 1.1 times a white blood cell concentration in the whole blood to a site of ischemic damage. Some embodiments relate to a method of reducing apoptosis in ischemia damaged tissue, comprising delivering a PRP composition comprising platelets derived from whole blood at a first concentration of at least about 1.1 times a platelet concentration in the whole blood and white blood cells derived from the whole blood at a second concentration of at least about 1.1 times a white blood cell concentration in the whole blood, wherein the white blood cells comprise neutrophils, wherein the neutrophil concentration is less than the neutrophil concentration in the whole blood and lymphocytes, wherein the lymphocyte concentration is 1.1 times lymphocyte concentration in the whole blood and monocytes, wherein the monocyte concentration is 1.1 times monocyte concentration in the whole blood to a site of ischemic damage.

Several embodiments described herein relate to a kit comprising a separating device for separation of whole blood into components for preparation of a platelet-containing composition, one or more collection devices, one or more means for sterilization, and a needle or catheter sufficient for injection of the platelet-containing composition. In some embodiments, the separation device provides a blood product enriched in platelets and depleted in neutrophils. In some embodiments, the separation device provides a blood product enriched in platelets and depleted in neutrophils to an amount of less than 80%, preferably less than 90%, more preferably less than 95% and more preferably less than 99% the levels found in whole blood. Several embodiments relate to a kit that further comprises a measurement device to test the efficacy of the platelet-containing composition for treatment of a pre-determined disease. Several embodiments relate to a kit used to treat a cardiovascular disease, lung disease, peripheral vascular disease, muscle, tendon, ligament, cartilage, bone, kidney, brain tissue and any other organ including pancreas, liver, a limb and skin. Some embodiments relate to a kit comprising one or more collection devices, wherein at least one collection device comprises one or more syringes, apheresis needles or other device for collecting blood from a patient. Some embodiments relate to a kit comprising a pH adjusting agent.

Several embodiments relate to method of treating a peripheral vascular disease comprising identifying ischemia in a limb of a patient and delivering a composition comprising platelets derived from whole blood at a first concentration of at least about 1.1 times a platelet concentration in the whole blood, white blood cells derived from the whole blood at a second concentration of at least a white blood cell concentration in the whole blood, wherein the white blood cells comprise neutrophils at a third concentration, wherein the third concentration is less than the neutrophil concentration in the whole blood and lymphocytes at a fourth concentration of at least 1.1 times a lymphocyte concentration in the whole blood; and monocytes at a fifth concentration of about 1.1 times a monocyte concentration in the whole blood to the patient to treat the ischemia. In some embodiments, the composition is prepared from the whole blood of the patient.

### BRIEF DESCRIPTION OF THE DRAWINGS

FIG. 1 shows the platelet concentration in whole blood and nonfractionated (RevaCor) PRP.

FIG. 2 shows the White Blood Cell (WBC) concentration in whole blood and nonfractionated (RevaCor) PRP.

FIG. 3 shows a graph comparing the cardiac ejection fraction of mice treated with nonfractionated (RevaCor) PRP to a control group treated with phosphate buffered saline (PBS) following myocardial ischemia-reperfusion injury.

FIG. 4 shows a photomicrograph of H&E and Trichrome staining of cardiac tissue collected from mice treated with nonfractionated (RevaCor) PRP or a control group treated with phosphate buffered saline (PBS) following myocardial ischemia-reperfusion injury.

FIG. 5 shows a western blot analysis of GAPDH, Caspase-3, Cleaved Caspase-3 and cleaved PARP expression in hypoxic endothelial cells, hypoxic endothelial cells treated with nonfractionated (RevaCor) PRP and in nonfractionated (RevaCor) PRP.

FIG. 6A shows the neutrophil concentration in nonfractionated (RevaCor) PRP and neutrophil-depleted (Vitakine) PRP.

FIG. 6B shows the platelet concentration in nonfractionated (RevaCor) PRP and neutrophil-depleted (Vitakine) PRP.

FIG. 7A shows ooclusion of the left anterior descending artery of a porcine subject

FIG. 7B shows a topographical injury distribution map.

FIG. 8 shows pathologic analysis of area at risk for myocardial injury between the cohorts.

FIG. 9A shows a graph comparing scar size at 21 days post myocardial injury between the control and nonfractionated (RevaCor) PRP treated cohorts.

FIG. 9B shows a graph comparing left ventricle ejection fraction at 21 days post myocardial injury between the control and neutrophil-depleted (Vitakine) PRP treated cohorts.

### DETAILED DESCRIPTION

### Overview

Blood is comprised of Red Blood Cells (RBC), White Blood Cells (WBC), Plasma, and Platelets. Platelet-rich plasma (PRP) is a fractionation of whole blood containing concentrated platelets and white blood cells and which may include high quantities of cytokines such as vascular endothelial growth factor (VEGF), transforming growth factor beta (TGF-β), and platelet-derived growth factor (PDGF). Platelets are responsible for blood clotting and when activated, release growth factors and other bioactive molecules which are involved in stimulating the healing of bone and soft tissue. For example, platelets release VEGF and basic fibroblast growth factor from alpha granules and adenosine diphosphate (ADP), adenosine triphosphate (ATP), and ionized calcium from dense granules. White blood cells (WBCs), also known as leukocytes, are involved in defending the body against both infectious disease and foreign materials. The two most common types of white blood cells are the lymphocytes and neutrophils. Lymphocytes secrete factors, lymphokines, which modulate the functional activities of many other types of cells and are often present at sites of chronic inflammation. Neutrophils, which are the most abundant white blood cell type in mammals, are recruited to the site of injury within minutes following trauma. Neutrophils form an essential part of the innate immune system, playing a role in inflammation.

PRP was initially used to enhance bone healing in cancer patients with jaw reconstruction and been extensively studied in other mesodermal tissues. PRP has been shown to stimulate cell proliferation, induce angiogenesis, and to safely and effectively enhance healing of tendon, ligament, muscle, and bone primarily by reparative cell signaling. When treated with PRP, skeletal muscle injuries recover full contractile function faster than in the absence of PRP treatment. PRP has also been shown to improve perfusion of ischemic tissue in a murine hindlimb ischemia model in addition to favorably modulating post-myocardial infarction remodeling in a rodent model. Further, investigators have reported PRP may alter the proliferation and expression of mesenchymal stem cells. Due to its excellent safety profile, PRP is widely used in sports medicine in the treatment of musculoskeletal injuries.

PRP is an interesting biologic treatment option not only because it contains increased concentrations of growth factors compared to whole blood, but also because it can be prepared and delivered at the point of care. Further, PRP is an autologous product with an extensive clinical use history and outstanding safety record. Embodiments described herein relate to PRP compositions and their use in treating ischemic injury, particularly ischemic injury to cardiac tissue. In cardiac tissue, PRP compositions may be used to treat ischemic tissue following an acute myocardial infarction (MI) to preserve myocardial tissue and promote re-growth. Additionally, PRP compositions may be used to reduce apoptosis, infarct size, decrease cardiac arrhythmias, and restore cell function. Not wishing to be bound by a particular theory, white blood cells may be critical to the formulation of PRP compositions as it has been shown that certain types of leukocytes participate in the healing response after an ischemia-reperfusion injury. (Linfert Trans. Rev. 2009)

Several embodiments described herein relate to neutrophil-depleted PRP compositions and their use in treating ischemic injury, particularly ischemic injury to cardiac tissue.

Treatment of Cardiac Ischemia with PRP Compositions

Some embodiments relate to the use of PRP compositions to reduce and/or eliminate ischemia-reperfusion induced apoptosis. Preclinical and early clinical cardiovascular investigations demonstrate that application of PRP compositions improves reperfusion, induces CD34⁺ cell migration, limits negative remodeling, and improves angina scores following ischemic damage to the myocardium. To study the effect of PRP on ischemia-induced apoptosis, an unfractionated PRP composition with increased platelets and white blood cells was tested in an in vitro ischemia-reperfusion model using cultured endothelial cells. Application of a PRP composition was found to decrease apoptosis of oxygen-deprived human microvascular endothelial cells (HMEC-1). Specifically, HMEC-1 cells receiving a PRP composition showed a significant decrease in the apoptosis markers caspase-3 (Casp-3), cleaved caspase-3 (Cl-Casp-3), and cleaved PARP (Cl-PARP) compared to untreated cells by Western blot analysis. These results indicate that PRP treatment limits apoptosis-associated tissue damage caused by ischemia-reperfusion injury. PRP compositions represent a novel treatment approach to limit apoptosis associated with a myocardial infarction.

Not wishing to be bound by a particular theory, PRP treatment may act to limit apoptosis via the release of growth factors and cytokines from platelets and via cell to cell interactions from the white blood cells within PRP that help preserve cells at risk for programmed cell death. Several investigators have previously reported that apoptosis may contribute to myocardial dysfunction by reducing the number of contractile cardiocytes. Apoptosis of cardiac non-myocytes can also contribute substantially to the progressive nature of failing myocardium through maladaptive remodeling. The function of the cardiac fibroblast is to maintain the extracellular matrix and provide a scaffold for myocytes to ensure proper heart form and function. Thus, by preserving cardiac fibroblasts and/or myocytes at risk for ischemic or apoptotic death, PRP helps limit the negative remodeling of cardiac tissue following ischemic injury. Further, not to be bound by a particular theory, stimulation of cardiac fibroblasts by the TGF-β within PRP may result in increased synthesis of fibullar collagen, proteoglycans and expression of contractile genes. Importantly, fibroblast growth factor and vascular endothelial growth factors (both present in PRP) are crucial for enhancing angiogenesis and promoting collateral formation after a myocardial injury.

Some embodiments relate to the use of unfractionated PRP having increased levels of platelets and WBCs compared to baseline levels in whole blood to improving cardiac output following ischemic injury. Several embodiments relate to PRP formulations having increased levels of platelets and white blood cells in combination for the treatment of specific conditions such as reducing the size of myocardial infarct after heart attack.

In a murine myocardial ischemia/reperfusion (I/R) model, application of a PRP composition containing an average concentration of platelets 5.06 times baseline and an average concentration of WBCs 3.6 time baseline had a protective effect. Mice treated with the PRP composition showed significant improvement in the left ventricle ejection fraction (LVEF) compared to untreated mice. Specifically, mice treated with the PRP composition had an 8% absolute and 28% relative improvement in left ventricular ejection fraction at 21 days after myocardial infarction. Further, histological analysis of the hearts taken from mice treated with PRP after myocardial infarction showed less scar tissue than the hearts of mice that did not receive PRP following myocardial infarction. Treatment with unfractionated PRP having increased levels of platelets and WBCs also decreased infarct size in swine subjected to myocardial ischemia-reperfusion injury. However, in the swine ischemia-reperfusion injury model, treatment with unfractionated PRP did not significantly improve LVEF compared to control.

The presence of scar tissue in heart can effect depolarization, leading to arrhythmia. People whose cardiac muscle wall thickness contained more than 25 percent scar tissue were approximately nine times more likely to test positive for a fast and dangerous heart rhythm known as ventricular arrhythmia. At least 30 percent of sudden cardiac deaths are due to arrhythmia. Not wishing to be bound by a particular theory, treatment of ischemia-damaged cardiac tissue with unfractionated platelet and WBC enriched PRP can reduce the instance of arrhythmia by decreasing formation of scar tissue at the infarct site.

Some embodiments relate to PRP formulations having increased levels of platelets and white blood cells in combination with depleted neutrophil levels. Such formulations are useful in treatment of specific disease conditions such as improvement in LVEF after myocardial infarction. Treatment of swine subjected to myocardial ischemia-reperfusion injury with neutrophil-depleted PRP significantly improved LVEF compared to control, however, treatment with neutrophil-depleted PRP did not significantly reduce infarct size.

The differing results of unfractionated PRP and neutraphil-depleted PRP treatment in the swine ischemia-reperfusion injury model suggests that both formulations have value in the treatment of myocardial injury. The differing bioactivity of unfractionated PRP and neutraphil-depleted PRP support specific cardiac applications in the treatment of acute myocardial infarction, congestive heart failure, and chronic angina.

### Compositions

The term “platelet-rich plasma” or “PRP” as used herein is a broad term which is used in its ordinary sense and is a concentration of platelets greater than the peripheral blood concentration suspended in a solution of plasma, or other excipient suitable for administration to a human or non-human animal including, but not limited to isotonic sodium chloride solution, physiological saline, normal saline, dextrose 5% in water, dextrose 10% in water, Ringer solution, lactated Ringer solution, Ringer lactate, Ringer lactate solution, and the like. PRP compositions may be an autologous preparation from whole blood taken from the subject to be treated or, alternatively, PRP compositions may be prepared from a whole blood sample taken from a single donor source or from whole blood samples taken from multiple donor sources. In general, PRP compositions comprise platelets at a platelet concentration that is higher than the baseline concentration of the platelets in whole blood. In some embodiments, PRP compositions may further comprises WBCs at a WBC concentration that is higher than the baseline concentration of the WBCs in whole blood. As used herein, baseline concentration means the concentration of the specified cell type found in the patient's blood which would be the same as the concentration of that cell type found in a blood sample from that patient without manipulation of the sample by laboratory techniques such as cell sorting, centrifugation or filtration. Where blood samples are obtained from more than one source, baseline concentration means the concentration found in the mixed blood sample from which the PRP is derived without manipulation of the mixed sample by laboratory techniques such as cell sorting, centrifugation or filtration.

In some embodiments, PRP compositions comprise elevated concentrations of platelets and WBCs and lower levels of RBCs and hemoglobin relative to their baseline concentrations. In some embodiments of PRP composition, only the concentration of platelets is elevated relative to the baseline concentration. Some embodiments of PRP composition comprise elevated levels of platelets and WBCs compared to baseline concentrations. In some embodiments, PRP compositions comprise elevated concentrations of platelets and lower levels of neutrophils relative to their baseline concentrations. Some embodiments of PRP composition comprise elevated levels of platelets and neutrophil-depleted WBCs compared to their baseline concentrations. In some embodiments of PRP, the ratio of lymphocytes and monocytes to neutrophils is significantly higher than the ratios of their baseline concentrations.

The PRP formulation may include platelets at a level of between about 1.01 and about 2 times the baseline, about 2 and about 3 times the baseline, about 3 and about 4 times the baseline, about 4 and about 5 times the baseline; about 5 and about 6 times the baseline, about 6 and about 7 times the baseline, about 7 and about 8 times the baseline, about 8 and about 9 times the baseline, about 9 and about 10 times the baseline, about 11 and about 12 times the baseline, about 12 and about 13 times the baseline, about 13 and about 14 times the baseline, or higher. In some embodiments, the platelet concentration may be between about 4 and about 6 times the baseline. Typically, a microliter of whole blood comprises at least 140,000 to 150,000 platelets and up to 400,000 to 500,000 platelets. The PRP compositions may comprise about 500,000 to about 7,000,000 platelets per microliter. In some instances, the PRP compositions may comprise about 500,000 to about 700,000, about 700,000 to about 900,000, about 900,000 to about 1,000,000, about 1,000,000 to about 1,250,000, about 1,250,000 to about 1,500,000, about 1,500,000 to about 2,500,000, about 2,500,000 to about 5,000,000, or about 5,000,000 to about 7,000,000 platelets per microliter.

The WBC concentration is typically elevated in PRP compositions. For example, the WBC concentration may be between about 1.01 and about 2 times the baseline, about 2 and about 3 times the baseline, about 3 and about 4 times the baseline, about 4 and about 5 times the baseline, about 5 and about 6 times the baseline, about 6 and about 7 times the baseline, about 7 and about 8 times the baseline, about 8 and about 9 times the baseline, about 9 and about 10 times the baseline, or higher. The WBC count in a microliter of whole blood is typically at least 4,100 to 4,500 and up to 10,900 to 11,000. The WBC count in a microliter of the PRP composition may be between about 8,000 and about 10,000; about 10,000 and about 15,000; about 15,000 and about 20,000; about 20,000 and about 30,000; about 30,000 and about 50,000; about 50,000 and about 75,000; and about 75,000 and about 100,000.

Among the WBCs in the PRP composition, the concentrations may vary by the cell type but, generally, each may be elevated. In some variations, the PRP composition may comprise specific concentrations of various types of white blood cells. The relative concentrations of one cell type to another cell type in a PRP composition may be the same or different than the relative concentration of the cell types in whole blood. For example, the concentrations of lymphocytes and/or monocytes may be between about 1.1 and about 2 times baseline, about 2 and about 4 times baseline, about 4 and about 6 times baseline, about 6 and about 8 times baseline, or higher. In some variations, the concentrations of the lymphocytes and/or the monocytes may be less than the baseline concentration. The concentrations of eosinophils in the PRP composition may be less than baseline, about 1.5 times baseline, about 2 times baseline, about 3 times baseline, about 5 times baseline, or higher.

In whole blood, the lymphocyte count is typically between 1,300 and 4,000 cells per microliter, but in other examples, the lymphocyte concentration may be between about 5,000 and about 20,000 per microliter. In some instances, the lymphocyte concentration may be less than 5,000 per microliter or greater than 20,000 per microliter. The monocyte count in a microliter of whole blood is typically between 200 and 800. In the PRP composition, the monocyte concentration may be less than about 1,000 per microliter, between about 1,000 and about 5,000 per microliter, or greater than about 5,000 per microliter. The eosinophil concentration may be between about 200 and about 1,000 per microliter elevated from about 40 to 400 in whole blood. In some variations, the eosinophil concentration may be less than about 200 per microliter or greater than about 1,000 per microliter.

In certain variations, the PRP composition may contain a specific concentration of neutrophils. The neutrophil concentration may vary between less than the baseline concentration of neutrophils to eight times than the baseline concentration of neutrophils. In some embodiments, the PRP composition may include neutrophils at a concentration of 50-70%, 30-50%, 10-30%, 5-10%, 1-5%, 0.5-1%, 0.1-0.5% of levels of neutrophils found in whole blood or even less. In some embodiments, neutrophil levels are depleted to 1% or less than that found in whole blood. In some variations, the neutrophil concentration may be between about 0.01 and about 0.1 times baseline, about 0.1 and about 0.5 times baseline, about 0.5 and 1.0 times baseline, about 1.0 and about 2 times baseline, about 2 and about 4 times baseline, about 4 and about 6 times baseline, about 6 and about 8 times baseline, or higher. The neutrophil concentration may additionally or alternatively be specified relative to the concentration of the lymphocytes and/or the monocytes. One microliter of whole blood typically comprises 2,000 to 7,500 neutrophils. In some variations, the PRP composition may comprise neutrophils at a concentration of less than about 1,000 per microliter, about 1,000 to about 5,000 per microliter, about 5,000 to about 20,000 per microliter, about 20,000 to about 40,000 per microliter, or about 40,000 to about 60,000 per microliter. In some embodiments, neutrophils are eliminated or substantially eliminated. Means to deplete blood products, such as PRP, of neutrophils is known and discussed in U.S. Pat. No. 7,462,268, which is incorporated herein by reference.

Several embodiments are directed to PRP compositions in which levels of platelets and white blood cells are elevated compared to whole blood and in which the ratio of monocytes and/or lymphocytes to neutrophils is higher than in whole blood. The ratio of monocytes and/or lymphocytes to neutrophils may serve as an index to determine if a PRP formulation may be efficaciously used as a treatment for a particular disease or condition. PRP compositions where the ratio of monocytes and/or lymphocytes to neutrophils is increased may be generated by either lowering neutrophils levels, or by maintaining neutrophil levels while increasing levels of monocytes and/or lymphocytes. Several embodiments relate to a PRP formulation that contains 1.01 times, or higher, baseline platelets in combination with a 1.01 times, or higher, baseline white blood cells with the neutrophil component depleted at least 1% from baseline.

In some embodiments, the PRP compositions may comprise a lower concentration of red blood cells (RBCs) and/or hemoglobin than the concentration in whole blood. The RBC concentration may be between about 0.01 and about 0.1 times baseline, about 0.1 and about 0.25 times baseline, about 0.25 and about 0.5 times baseline, or about 0.5 and about 0.9 times baseline. The hemoglobin concentration may be depressed and in some variations may be about 1 g/dl or less, between about 1 g/dl and about 5 g/dl, about 5 g/dl and about 10 g/dl, about 10 g/dl and about 15 g/dl, or about 15 g/dl and about 20 g/dl. Typically, whole blood drawn from a male patient may have an RBC count of at least 4,300,000 to 4,500,000 and up to 5,900,000 to 6,200,000 per microliter while whole blood from a female patient may have an RBC count of at least 3,500,000 to 3,800,000 and up to 5,500,000 to 5,800,000 per microliter. These RBC counts generally correspond to hemoglobin levels of at least 132 g/L to 135 g/L and up to 162 g/L to 175 g/L for men and at least 115 g/L to 120 g/L and up to 152 g/L to 160 g/L for women.

In some embodiments, PRP compositions contain increased concentrations of growth factors and other cytokines. In several embodiments, PRP compositions may include increased concentrations of one or more of: platelet-derived growth factor, transforming growth factor beta, fibroblast growth factor, insulin-like growth factor, insulin-like growth factor 2, vascular endothelial growth factor, epidermal growth factor, interleukin-8, keratinocyte growth factor, and connective tissue growth factor. In some embodiments, the platelets collected in PRP are activated by thrombin and calcium chloride to induce the release of these growth factors from alpha granules.

In some embodiments, a PRP composition is activated exogenously with thrombin and/or calcium to produce a gel that can be applied to an area to be treated. The process of exogenous activation, however, results in immediate release of growth factors. Other embodiments relate to activation of PRP via in vivo contact with collagen containing tissue at the treatment site. The in vivo activation of PRP results in slower growth factor release at the desired site.

### Methods of Making

The PRP composition may comprise a PRP derived from a human or animal source of whole blood. The PRP may be prepared from an autologous source, an allogenic source, a single source, or a pooled source of platelets and/or plasma. To derive the PRP, whole blood may be collected, for example, using a blood collection syringe. The amount of blood collected may depend on a number of factors, including, for example, the amount of PRP desired, the health of the patient, the severity or location of the tissue damage and/or the MI, the availability of prepared PRP, or any suitable combination of factors. Any suitable amount of blood may be collected. For example, about 1 cc to about 150 cc of blood or more may be drawn. More specifically, about 27 cc to about 110 cc or about 27 cc to about 55 cc of blood may be withdrawn. In some embodiments, the blood may be collected from a patient who may be presently suffering, or who has previously suffered from, connective tissue damage and/or an MI. PRP made from a patient's own blood may significantly reduce the risk of adverse reactions or infection.

In an exemplary embodiment, about 55 cc of blood may be withdrawn into a 60 cc syringe (or another suitable syringe) that contains about 5 cc of an anticoagulant, such as a citrate dextrose solution. The syringe may be attached to an apheresis needle, and primed with the anticoagulant. Blood (about 27 cc to about 55 cc) may be drawn from the patient using standard aseptic practice. In some embodiments, a local anesthetic such as anbesol, benzocaine, lidocaine, procaine, bupivicaine, or any appropriate anesthetic known in the art may be used to anesthetize the insertion area.

The PRP may be prepared in any suitable way. For example, the PRP may be prepared from whole blood using a centrifuge. The whole blood may or may not be cooled after being collected. Isolation of platelets from whole blood depends upon the density difference between platelets and red blood cells. The platelets and white blood cells are concentrated in the layer (i.e., the “buffy coat”) between the platelet depleted plasma (top layer) and red blood cells (bottom layer). For example, a bottom buoy and a top buoy may be used to trap the platelet-rich layer between the upper and lower phase. This platelet-rich layer may then be withdrawn using a syringe or pipette. Generally, at least 60% or at least 80% of the available platelets within the blood sample can be captured. These platelets may be resuspended in a volume that may be about 3% to about 20% or about 5% to about 10% of the sample volume.

In some examples, the blood may then be centrifuged using a gravitational platelet system, such as the Cell Factor Technologies GPS System® centrifuge. The blood-filled syringe containing between about 20 cc to about 150 cc of blood (e.g., about 55 cc of blood) and about 5 cc citrate dextrose may be slowly transferred to a disposable separation tube which may be loaded into a port on the GPS centrifuge. The sample may be capped and placed into the centrifuge. The centrifuge may be counterbalanced with about 60 cc sterile saline, placed into the opposite side of the centrifuge. Alternatively, if two samples are prepared, two GPS disposable tubes may be filled with equal amounts of blood and citrate dextrose. The samples may then be spun to separate platelets from blood and plasma. The samples may be spun at about 2000 rpm to about 5000 rpm for about 5 minutes to about 30 minutes. For example, centrifugation may be performed at 3200 rpm for extraction from a side of the separation tube and then isolated platelets may be suspended in about 3 cc to about 5 cc of plasma by agitation. The PRP may then be extracted from a side port using, for example, a 10 cc syringe. If about 55 cc of blood may be collected from a patient, about 5 cc of PRP may be obtained.

As the PRP composition comprises activated platelets, active agents within the platelets are released. These agents include, but are not limited to, cytokines (e.g., IL-1B, IL-6, TNF-A), chemokines (e.g., ENA-78 (CXCL5), IL-8 (CXCL8), MCP-3 (CCL7), MIP-1A (CCL3), NAP-2 (CXCL7), PF4 (CXCL4), RANTES (CCL5)), inflammatory mediators (e.g., PGE2), and growth factors (e.g., Angiopoitin-1, bFGF, EGF, FGF, HGF, IGF-I, IGF-II, PDAF, PDEGF, PDGF AA and BB, TGF-.beta. 1, 2, and 3, and VEGF).

The PRP composition may be delivered as a liquid, a solid, a semi-solid (e.g., a gel), an inhalable powder, or some combination thereof. When the PRP is delivered as a liquid, it may comprise a solution, an emulsion, a suspension, etc. A PRP semi-solid or gel may be prepared by adding a clotting agent (e.g., thrombin, epinephrine, calcium salts) to the PRP. The gel may be more viscous than a solution and therefore may better preserve its position once it is delivered to target tissue. In some embodiments, the PRP composition is delivered without a clotting agent.

In some instances, it may be desirable to deliver the PRP composition as a liquid and have it gel or harden in situ. For example, the PRP compositions may include, for example, collagen, cyanoacrylate, adhesives that cure upon injection into tissue, liquids that solidify or gel after injection into tissue, suture material, agar, gelatin, light-activated dental composite, other dental composites, silk-elastin polymers, Matrigel® gelatinous protein mixture (BD Biosciences), hydrogels and/or other suitable biopolymers. Alternatively, the above mentioned agents need not form part of the PRP mixture. For example, the above mentioned agents may be delivered to the target tissue before or after the PRP has been delivered to the target tissue to cause the PRP to gel. In some embodiments, the PRP composition may harden or gel in response to one or more environmental or chemical factors such as temperature, pH, proteins, etc.

The PRP may be buffered using an alkaline buffering agent to a physiological pH. The buffering agent may be a biocompatible buffer such as HEPES, TRIS, monobasic phosphate, monobasic bicarbonate, or any suitable combination thereof that may be capable of adjusting the PRP to physiological pH between about 6.5 and about 8.0. In certain embodiments, the physiological pH may be from about 7.3 to about 7.5, and may be about 7.4. For example, the buffering agent may be an 8.4% sodium bicarbonate solution. In these embodiments, for each cc of PRP isolated from whole blood, 0.05 cc of 8.4% sodium bicarbonate may be added. In some embodiments, the syringe may be gently shaken to mix the PRP and bicarbonate.

As noted above, the PRP composition may comprise one or more additional agents, diluents, solvents, or other ingredients. Examples of the additional agents include, but are not limited to, thrombin, epinephrine, collagen, calcium salts, pH adjusting agents, materials to promote degranulation or preserve platelets, additional growth factors or growth factor inhibitors, NSAIDS, steroids, anti-infective agents, and mixtures and combinations of the foregoing.

In some embodiments, the PRP compositions may comprise a contrast agent for detection by an imaging technique such as X-rays, magnetic resonance imaging (MRI), or ultrasound. Examples of such contrast agents include, but are not limited to, X-ray contrast (e.g., IsoVue), MRI contrast (e.g., gadolinium), and ultrasound contrast.

### Neutrophil-Depleted PRP

Neutrophils, as stated above, are a type of white blood cell found commonly in whole blood. They are attracted to dyes that do not have a positive or negative charge. Therefore, they are neutral. Platelets and other white blood cells such as monocytes and lymphocytes, conversely, have a negative surface membrane charge. Importantly, also, neutrophils are less deformable than red blood cells (also known as erythrocytes). Because the neutrophils are less capable of changing shape and are relatively large, they take longer to pass through either a tight radius of curvature or through an area of constriction within a blood vessel. This partially explains why neutrophils can get stuck in the tight pulmonary circulation and cause lung damage.

Neutrophils may be separated from PRP or whole blood on the basis of their size, shape and charge. Neutrophils are larger, less deformable and neutrally charged relative to other blood components which are smaller, deformable and negatively charged. Thus, by forcing a blood, platelet or platelet rich plasma fraction through a narrow, twisted and/or charged environment, neutrophils are preferentially removed from other blood components. Methods and devices for separation of Neutrophils from whole blood and PRP are described in U.S. Pat. No. 7,462,268 to Allan Mishra, incorporated in its entirety by reference herein.

In some embodiments, the neutrophils have been depleted by at least 5%, in some embodiments, the neutrophils are depleted by at least 10%, in some embodiments, the neutrophils are depleted by at least 15%, in some embodiments, the neutrophils are depleted by at least 20%, in some embodiments, the neutrophils are depleted by at least 25%, in some embodiments, the neutrophils are depleted by at least 30%, in some embodiments, the neutrophils are depleted by at least 35%, in some embodiments, the neutrophils are depleted by at least 40%, in some embodiments, the neutrophils are depleted by at least 45%, in some embodiments, the neutrophils are depleted by at least 50%, in some embodiments, the neutrophils are depleted by at least 55%, in some embodiments, the neutrophils are depleted by at least 60%, in some embodiments, the neutrophils are depleted by at least 65%, in some embodiments, the neutrophils are depleted by at least 70%, in some embodiments, the neutrophils are depleted by at least 75%, in some embodiments, the neutrophils are depleted by at least 80%, in some embodiments, the neutrophils are depleted by at least 85%, in some embodiments, the neutrophils are depleted by at least 90%, in some embodiments, the neutrophils are depleted by at least 95%, in some embodiments, the neutrophils are depleted by at least 95%. In some embodiments, the neutrophils in the platelet rich plasma are substantially removed.

### Methods of Testing

In some variations, the PRP composition may be analyzed and/or modified prior to delivery to the patient. The PRP composition may be modified based on, for example, the condition to be treated, an initial complete blood count, a genetic profile of the patient, and other suitable factors.

In some embodiments, a patient's genetic profile is determined. The PRP composition of healthy individuals having the same or similar genetic profile is determined. A PRP composition is prepared in which components are matched to the PRP of the healthy individual having the same genetic profile. The modified PRP composition is administered to the patient to treat the disease or condition.

In some embodiments, the PRP composition of a patient, successfully recovering from a disease or condition may be used as a model to prepare a PRP composition to administer to a patient diagnosed with the same disease or condition. In other words, the PRP composition is first enriched in components which are effective in treating the disease based upon recovered or recovering individuals. The modified PRP composition is then administered to the patient suffering from the disease.

The PRP, or a portion of the PRP, may be placed into an automated blood analyzer that performs a compete blood count (CBC). As part of the CBC, the automated blood analyzers typically return a count of the number of platelets, WBCs, and RBCs present in the sample. The WBC count may further include counts of lymphocytes, monocytes, basophils, neutrophils, and/or eosinophils. Examples of blood analyzers that may be used include, but are not limited to, Beckman Coulter LH series, Sysmex XE-2100, Siemens ADVIA 120 & 2120, and the Abbott Cell-Dyn series.

It is believed that the effectiveness of treatments using PRP may be at least partially dependent on the genetic profile of the patient. The whole blood of a patient may be tested before and/or after generating the PRP composition to determine if the PRP composition is likely to affect the ability of the tissue to regenerate. Once the PRP has been determined to be useful, it may be delivered to the patient.

In certain variations, one or more genetic markers of a patient's DNA, mRNA, proteins, or the like may be evaluated prior to, during, and/or after delivery of the PRP composition. The patient's DNA, or other biomarkers, is typically captured via a sample such as blood, saliva, or other suitable body fluid or body tissue. The sample may be tested for genetic markers that are correlatable to the effectiveness of treatments using the PRP composition. In some instances, the identified genetic markers may be detectable using a genetic tool such as a gene chip or other genetic expression technology. In some instances, the genes that may be tested for include, but are not limited to, collagen type I (COL1A1), collagen type III (COL3A1), cartilage oligomeric matrix protein (COMP), matrix metalloproteinase-3 (MMP-3), and matrix metalloproteinase-13 (MMP-13). Such genetic tools can be used to measure changes in expression levels, or to detect single nucleotide polymorphisms (SNPs) which may be associated with a disease condition. Many gene chips are commercially available including the Affymetrix Gene Chip®, the Applied Microarrays CodeLink® arrays, and the Eppendorf DualChip & Silverquant®.

In some variations, the genetic tool may be analyzed to determine if the patient is likely to respond (favorably or unfavorably) to the PRP composition and/or to subsequent treatments. In certain variations, the PRP composition may be tested at a range of pH values and/or the pH of the PRP may be modified based at least in part on the genetic profile. In some instances, various genetic profiles may be associated with specific concentrations (or ranges of concentrations) as being more or less effective than other concentrations for various components of the PRP composition. The response to the PRP composition may be slowing or halting of cardiac apoptosis, anti-arrhythmia effects, or otherwise decrease risks associated with reperfusion therapy.

If the CBC returned by the automated blood analyzer is not within specified ranges, the PRP composition may be modified using a filtration device and/or cell sorter. The filtration device may use vacuum and/or gravity to remove a portion of the platelet, WBCs, and/or RBCs. In some variations, a cell sorter may receive a CBC input from an automated blood analyzer and/or a gene chip reader. A user may select or confirm one or more modifications to be made to the PRP composition. Of course, the cell sorter may be used with whole blood, portions of whole blood, and/or PRP. The cell sorter may sort the PRP composition based on electric charge, density, size, deformation, fluorescence, or the like. Examples of cell sorters include the BD FACSAria® cell sorter, the Cytopeia InFlux® cell sorter, those manufactured by Beckman Coulter, the Cytonome Gigasort® cell sorter, and the like.

### Methods of Use

The PRP composition may be delivered at any suitable dose. In some embodiments, the dose may be between about 1 cc and about 3 cc, between about 3 cc and about 5 cc, between about 5 cc and about 10 cc, between about 10 cc and about 20 cc, or more. The dose may be delivered according to a medical procedure (e.g., at specific points in a procedure) and/or according to a schedule. For example, prior to an elective cardioversion, the PRP composition may be delivered about 24 hours, about 12 hours, about 6 hours, about 2 hours, and/or about 1 hour before the procedure begins.

In some embodiments, the PRP composition may be delivered to tissue damaged by ischemia or reperfusion injury. The list of tissues includes, but is not limited to, the heart, ischemic limbs, ischemic or damaged organs including the brain and skin. The PRP composition may be delivered to an individual in need thereof by injection using a syringe or catheter. The PRP composition may also be delivered via delivery device such as a dermal patch, a spray device, sutures, stents, screws, plates, or some other implantable medical device such as bioresorbable tissue patch. The PRP composition may be used as a coating or incorporated into the delivery device. The PRP delivery device may be incubated with PRP prior to use. Incubation times may be from a few seconds up to any convenient time such as a few seconds to hours before use, such as less than 1 minute, 5-10 minutes, 10 minutes to an hour, 1-3 hours, 4-12 hours, 13-24 hours, 1-3 days, or 3-31 days. PRP compositions may be used in conjunction with an ointment, bone graft, or drug.

The PRP alone or in combination with a delivery device may be conveniently stored in an appropriate chamber. In some embodiments, the PRP and/or PRP combined delivery device may be stored frozen and/or under reduced oxygen concentration or increased oxygen concentration, low and/or high pH, low and/or high pressure, low and/or high UV or other light conditions, low and/or high temperature. Storage times may vary from such as less than 1 minute, 5-10 minutes, 10 minutes to an hour, 1-3 hours, 4-12 hours, 13-24 hours, 1-3 days, 3-31 days, or 1-12 months or 1-5 years. The PRP composition alone or in combination with the delivery device may then be used clinically as appropriate.

In one exemplary embodiment, a platelet rich plasma composition is prepared and combined with a stent in an appropriate low oxygen chamber for 1-30 minutes, preferably about 10 minutes. The chamber is then exposed to ultraviolet light for a brief period of time, such as 1-60 seconds, 1-5 minutes, or 5-15 minutes. The stent is then removed from the chamber and implanted into a patient. It is expected that this chamber will improve the biologic activity of the platelet rich plasma and or device.

The site of delivery of the PRP composition is typically at or near the site of tissue damage. The site of tissue damage may be determined by well-established methods including imaging studies and patient feedback or a combination thereof. The preferred imaging method used may be determined based on the tissue type. Commonly used imaging methods include, but are not limited to MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), laser optical imaging NOGA mapping and ultrasound techniques. The patient may also assist in locating the site of tissue injury or damage by pointing out areas of particular pain and/or discomfort.

The PRP compositions described herein may also be used to treat peripheral vascular disease, strokes or other ischemic areas such as a kidney that was damaged. PRP compositions could also be used as a primary or secondary treatment for pulmonary disease.

In some examples, a PRP composition may be used to treat a patient diagnosed with an acute myocardial infarction or ischemic heart disease. Treatment with the PRP composition may occur in the field or in the emergency room setting. Criteria for PRP composition treatment may include positive cardiac markers, ST-elevations, or new wall motion abnormalities identified on echocardiogram, for example. The decision to treat with a PRP composition, and the treatment location(s), may depend upon one or more characteristics of the myocardial infarction. For example, a myocardial infarction may be characterized as a ST-elevation myocardial infarction (STEMI) or non-ST-elevation myocardial infarction (NSTEMI), a Q-wave or non-Q-wave myocardial infarction, and whether they are subendocardial or transmural. Myocardial infarctions may also be characterized anatomically by cardiac wall region and/or the suspected blockage site in the cardiac vasculature. Myocardial infarctions may also be characterized as anterior, lateral, inferior, posterior, septal, or right-ventricular in location, and may involve disease or blockage of the left-anterior descending, left circumflex, left main, posterior-descending and right coronary arteries, for example.

In some embodiments, timing of the PRP preparation and application may be based upon other treatments that are indicated in a patient with a myocardial infarction or ischemic heart disease. In some embodiments, a PRP composition may be prepared and delivered before, during, and/or after reperfusion therapy is performed to treat an acute myocardial infarction, a previous myocardial infarction, or ischemic heart disease. Reperfusion therapies may include thrombolytic therapy (such as heparin, TPA and or other pharmacologic agents), angioplasty, stenting (including bare metal stents and drug-eluting stents) or coronary artery bypass graft (CABG) surgery. In some instances, reperfusion therapy may be associated with an increased risk of an arrhythmia, including sudden death. Also, it is believed that the etiology of reperfusion arrhythmias or reperfusion arrhythmia risk may be different from the arrhythmia etiologies associated with the myocardial infarction itself. For example, some reperfusion arrhythmias may be caused by triggered activity and/or re-entry. In some embodiments, PRP composition is prepared before or at the start of a reperfusion procedure, but not used unless an arrhythmia occurs during the procedure. In other embodiments, the patient may be prophylactically pre-treated with a PRP composition before reperfusion occurs, e.g., before guidewire passage across an occlusion, stent positioning, stent expansion, or reestablishment of coronary flow through a bypass segment.

In some embodiments, the PRP composition is injected into or near an infarct site. The location of the infarct site may be determined or approximated using various techniques. For example, in some variations, diagnostic procedures such as an electrophysiology study or an electrical mapping study of the heart may be used. In other variations, one or more imaging technologies such as MRI, X-ray, CT scan, Positron Emission tomography (PET), Single Photon Emission Computed Tomography (SPECT), Electrical Impedance Tomography (EIT), Electrical Source Imaging (ESI), Magnetic Source Imaging (MSI), NOGA mapping, laser optical imaging and ultrasound techniques may be used. Other technologies and approaches that may be used include visual inspection during open chest surgical procedures, localized blood flow determinations, local electrical and structural activity, nuclear cardiology, echocardiography, echocardiographic stress test, coronary angiography, magnetic resonance imaging (MRI), computerized tomography (CT) scans, and ventriculography.

PRP compositions that are formulated as gels or other viscous fluids may be difficult to deliver via a needle or syringe. Thus, in variations where the use of a needle or syringe is desirable, a gelling and/or hardening agent may be optionally added to the PRP composition in situ. One or more needles or catheters may be configured to deliver the PRP composition and/or the gelling or hardening agent simultaneously, or substantially simultaneously, to the cardiac tissue. For example, if a needle is used to deliver the PRP composition, the needle may comprise a plurality of lumens through which the PRP composition and the agent separately travel. Alternatively or additionally, separate needles may be used to deliver the components to the tissue at the same time or one after the other.

The PRP composition may be delivered minimally invasively and/or surgically. For example, the PRP composition may be delivered to the heart using a catheter inserted into the patient via the femoral vein or artery, the internal jugular vein or artery, or any other suitable vein or artery. The PRP composition may be delivered along with one or more medical devices, instruments, or agents to treat the MI and/or other cardiac conditions.

To deliver a PRP composition to the ischemic tissue, a physician may use one of a variety of access techniques. These include surgical (e.g., sternotomy, thoracotomy, mini-thoracotomy, sub-xiphoidal) approaches, endoscopic approaches (e.g., intercostal and transxiphoidal) and percutaneous (e.g., transvascular, endocardial, and pericardial) approaches. Once access has been obtained, the composition may be delivered via epicardial, endocardial, or transvascular approaches. The composition may be delivered to the cardiac wall tissue or cardiac vessels in one or more locations. This includes intra-myocardial, sub-endocardial, and/or sub-epicardial administration.

Upon gaining access to the ischemic tissues of the heart, the delivery device may be inserted through any appropriate vessel. The distal end of the delivery device may be then placed against the surface of the myocardium and one or more needles may be advanced into tissue. Following delivery of one or more components of the PRP composition, the needles, if any, may be retracted. Mapping or guidance systems that rely upon voltage, ultrasound or pressure in addition to other systems may be used in combination with injection. The delivery device may then be repositioned for additional delivery of one or more components of the composition or may be removed from the patient. Incisions may then be closed using standard techniques.

The delivery system may deliver the components of the PRP composition in a prescribed ratio (e.g., a ratio of the lymphocytes and the neutrophils). The prescribed ratio may be calculated beforehand or determined on an ad hoc basis once delivery begins. To deliver the components in the prescribed ratio, the delivery device may include one or more gears having a corresponding gear ratio, one or more lumens having a proportional lumen size, or any other suitable mechanism. Some delivery devices may include one or more mixing chambers. The multiple components may be delivered using separate delivery devices or may be delivered one after the other using the same delivery device.

The delivery devices may be advanced through a vessel adjacent to the ischemic tissue to be treated. The PRP composition may be injected directly into the ischemic tissue using a needle and/or a needle-tip catheter. The PRP composition may alternatively or additionally be infused into the vessel.

When the PRP compositions are delivered using one or more catheters, any suitable catheter may be used. For example, the catheters may include one or more lumens and staggered or flush tips. The catheters may include needles or other devices (e.g., imaging devices) located at the distal end, and plungers or any other control located at the proximal end. The catheters and/or other delivery devices may have differently sized lumens to deliver multiple components of the PRP composition in the prescribed ratio. When catheters are used, a physician may navigate to the heart using one of the routes known for accessing the heart through the vasculature, including but not limited to navigation to a heart chamber for epicardial, endocardial, and/or transvascular delivery of the PRP composition.

Endocardial delivery of the PRP composition may comprise accessing a treatment site, for example, in the left ventricle of a heart, using a delivery device advanced percutaneously in an anterograde approach through the superior vena cava or inferior vena cava into the right ventricle. The delivery device may be passed through the interatrial septum into the left atrium and then into the left ventricle to reach treatment site. Alternatively, the device may be advanced using a transseptal procedure, e.g., through the intraventricular septum into the left ventricle. In another embodiment, the PRP composition may be injected directly into the intraventricular septum from the right ventricle. An alternative endocardial delivery method may comprise accessing the treatment site using a delivery device advanced percutaneously in a retrograde approach through the aorta into the left atrium and then into the left ventricle.

Transvascular delivery of compositions may comprise passing the delivery device through the coronary sinus into the cardiac venous system via the cardiac veins and, if needed, leaving the veins by tracking through myocardial tissue. An alternative transvascular delivery method comprises accessing a treatment site through the aorta into a coronary artery to reach treatment site.

A practitioner may make multiple deliveries into various locations using a single device, make multiple deliveries into various locations using multiple devices, make a single delivery to a single location using a single device, or make a single delivery to a single location using multiple devices. The deliver devices may include at least one reusable needle or catheter. Some embodiments may include delivery devices having an automated dosing system (e.g., a syringe advancing system). The automated dosing system may allow each dose to be pre-determined and dialed in (may be variable or fixed). In some embodiments, an iontophoresis device may be used to deliver the PRP composition into the ischemic tissue.

It may be desirable to deliver the PRP composition to the ischemic tissues while avoiding coincidental delivery to other cardiac tissues or other locations adjacent to the heart. For example, the PRP composition may gel or harden upon delivery to prevent migration. In other embodiments, the PRP compositions may be delivered without a gelling agent/activator such as thrombin. In some variations, a balloon catheter may be placed in the coronary sinus and inflated during delivery until the PRP composition has solidified or at least partially immobilized. Other variations may include a pressure control system on the delivery device to prevent pressure-driven migration of the PRP composition. Backbleed may also be prevented by keeping the needle in place for several seconds (e.g., about 5 to about 30 seconds, or about 5 to about 120 seconds) following an injection.

Sensors may be used to direct the delivery device to a desired location and/or to deliver the PRP composition. For example, real-time recording of electrical activity (e.g., an ECG), pH, oxygenation, metabolites such as lactic acid, CO₂, or the like may be used. The sensors may be one or more electrical sensors, fiber optic sensors, chemical sensors, imaging sensors, structural sensors, and/or proximity sensors that measure conductance. The sensors may be incorporated into the delivery device or be separate from the delivery device. In some embodiments, the sensors may sense and/or monitor such things as needle insertion depth, blood gas, blood pressure or flow, hemocrit, light, temperature, vibration, voltage, electric current, power, and/or impedance. The sensors may include one or more imaging systems and may be coupled to any appropriate output device, for example, a LCD or CRT monitor which receives and displays information.

The total volume of the PRP composition delivered to the patient may be based on the size of the heart, the amount of the affected ischemic tissue, and/or the desired outcome of the procedure. For example, the total volume of composition injected may be less than 15000 μL.

The number of delivery sites in the heart may be based on the type and location of the infarct(s), the desired location of the PRP composition, and the distance separating the desired locations. The number of delivery sites may range from about 1 to about 25 sites. The distance separating delivery sites may vary based on the desired volume of PRP to be delivered per delivery site, the desired total volume to be delivered, and/or the condition of the ischemic tissue. At the delivery site, the PRP composition may be injected, infused, or otherwise disposed at or adjacent to the ischemic tissue. The PRP composition may also be infused into the vasculature (i.e., vessels) upstream of the target site, so that it will flow towards the affected ischemic tissue.

The location of the delivery sites may vary based on the size and shape of the ischemic tissue, and the desired extent of the treatment of the tissue. For example, the PRP composition may be delivered into the ischemic tissue, and/or into the tissue that bordering the ischemic tissue. Similarly, the composition may be delivered to any combination of the regions of ischemic tissue and other cardiac tissue.

The timing of PRP delivery relative to an acute MI may be based on the severity of the infarction, the extent of the ischemic tissue, the condition of the patient, and the progression of any concurrent MI or arrhythmia treatments. The PRP composition may be delivered at any suitable time. For example, it may be delivered immediately after the onset of an MI, within one hour of an MI, one to eight hours following an MI, or three to four days after an MI after clinical stabilization of the patient when it is safer for the patient to undergo a separate procedure. Treatment may also be done later. The timing may be based upon the level of caspase-3 in the blood. In some variations, the composition is delivered about one week, about 1 to about 3 weeks, about 1 to about 6 months, or even up to or more than about 1 year after the MI. Treatment may be done for patients with congestive heart failure, cardiomyopathy or other heart disorders. Other times for injecting compositions into the ischemic tissue are also contemplated, including prior to any potential MI, and immediately upon finding an area of ischemic tissue. Of course, compositions may be injected into the ischemic tissue years after an MI.

As mentioned previously, a PRP composition may additionally or alternatively be used in other cardiac procedures. These cardiac procedures may include anti-arrhythmia procedures, procedures to correct congenital heart defects, or other pathologies. Examples of other cardiac procedures include, but are not limited to, angioplasty, coronary artery bypass, Minimally Invasive Direct Coronary Artery Bypass (MIDCAB), off-pump coronary artery bypass, Totally Endoscopic Coronary Artery Bypass (TECAB), aortic valve repair, aortic valve replacement, mitral valve repair, mitral valve replacement, Ross procedure, Bentall procedure, pulmonary thromboendarterectomy, transmyocardial revascularization (TMR), valve-sparing aortic root replacement, cardiomyoplasty, Dor procedure, heart transplantation, septal myectomy, ventricular reduction, pericardiocentesis, pericardiectomy, atrial septostomy, Blalock-Taussig shunt procedure, Fontan procedure, Norwood procedure, Rastelli procedure, Maze procedure (Cox maze and minimaze), and/or pacemaker insertion. The PRP composition may used to prevent an arrhythmia associated with reperfusion of the cardiac tissue during any of the above procedures. As is known, reperfusion may cause a spontaneous arrhythmia to occur after cardiac surgery.

The PRP composition may be used alone and or in combination with other therapies including, but not limited to, stems cells (embryonic or adult), cord blood, drugs, genetically engineered molecules, or other bioactive substances. In some embodiments, the PRP composition may be provided on or incorporated in a polyester or a poly(propylene) (Marlex) mesh that is sutured on or wrapped around an infract site to prevent negative LV remodeling and LV dilation associated with ischemic damage of cardiac tissue. In some embodiments, a composition comprising PRP and cardiomyocytes are delivered to an area affected by ischemic damage. In some embodiments, PRP composition may be provided to an infarct site in conjunction with one or more of fetal cardiomyocytes, embryonic stem cells, bone marrow cells, induced pluripotent stem cells, and cardiomyocytes derived from induced pluripotent stem cells. In some embodiments, a biomaterial scaffold comprised of gelatin, alginate, collagen type 1 and Matrigel, polyglycolide, collagen, fibrin, or self-assembling peptides is provided.

In some embodiments, PRP may be used to treat any lung disease. Examples of lung disease include, but are not limited to: Acute Respiratory Distress Syndrome (ARDS), Alpha-1-Antitrypsin Deficiency, Asbestos-Related Lung Diseases, Asbestosis, Asthma, Bronchiectasis, Bronchitis, Bronchopulmonary Dysplasia (BPD), Chronic Bronchitis (see COPD), Chronic Obstructive Pulmonary Disease (COPD), Collapsed Lung (see Atelectasis), Cough, Cystic Fibrosis, Emphysema (see COPD), Hemothorax, Idiopathic Pulmonary Fibrosis, Infant Respiratory Distress Syndrome (Respiratory Distress Syndrome in Infants), LAM (Lymphangioleiomyomatosis), Lung Transplant, Pleural Effusion, Pleurisy and Other Pleural Disorders, Pneumonia, Pneumonoconiosis, Pneumothorax (see Pleurisy and Other Disorders of the Pleura), Pulmonary Embolism, Pulmonary Arterial Hypertension, Pulmonary Fibrosis (see Idiopathic Pulmonary Fibrosis), Respiratory Distress Syndrome in Infants, Respiratory Failure, Sarcoidosis, Tracheostomy, and Ventilator/Ventilator Support. In some embodiments, PRP compositions are delivered directly to the lung via bronchoscopy or delivering indirectly to the lung via the heart or blood vessel. Measurements of tissue perfusion or function may be done to evaluate the efficacy of the treatment.

In some embodiments PRP is useful in treatment of disease and conditions in a variety of tissues including but not limited to heart, lung, liver, kidney, brain, spinal cord, muscle, tendon, bone, skin, ligaments and any other body cell or tissue. Rotator Cuff Tendinitis or Tear, Rotator Cuff Impingement Syndrome or Bursitis, Bicipital Tendinitis, labrum tears, arthritis, instability DeQuervaine's Tenosynovitis, arthritis, other wrist or finger tendinitis, ligament tears or dysfunction of the fingers Illiotibial Band Tendinitis (ITB Syndrome), Psoas Tendinitis and bursitis, Greater Trochanteric Bursitis, Hip labrum tears, Piriformis Syndrome, Sacroiliac Joint Dysfunction, arthritis Patellar Tendinitis, Patellar Femoral Syndrome, chondromalacia patella, partially torn or strained major ligaments of knee (ACL/LCL/MCL), meniscus tears, arthritis, patellar instability Achilles Tendinitis, Peroneal Tendinitis, arthritis, recurrent ankle sprains, other foot or ankle tendinitis Whiplash injuries, headaches related to the neck, arthritis Facet joint arthritis, rib problems, and pain associated with scoliosis. In some embodiments, PRP compositions may be use to treat disogenic spine pain or disorders alone or in combination with other treatments.

### Kits

Kits may include any device, component, or combination of devices and/or components described herein. For example, the kits may include one or more preparation devices, one or more delivery devices, one or more collection devices, and/or instructions for use. The one or more preparation devices may be for preparing PRP and may comprise a centrifuge, for example. The one or more delivery devices may be configured to deliver a PRP composition comprising the PRP to damaged connective tissue or to a region of the heart. The one or more collection devices may comprise one or more syringes, apheresis needles, or other devices for collecting blood from a patient. The components of the kit may be provided in a sterile condition with an expiration date. The kits may comprise one or more single-use components. Instructions may be in written or pictograph form, or may be on recorded media including audio tape, audio CD, video tape, DVD, CD-ROM, or the like.

Some embodiments relate to kits for making a PRP composition. For example, a kit may comprise one or more components to draw blood, one or more tubes to fractionate the blood in a centrifuge and/or other separation devices. In some embodiments, a syringe and tourniquet are provided to draw blood for preparation of the PRP composition. One skilled in the art would be able to determine the amount of blood to withdraw for treatment of a specific injury. In several embodiments, 20-150 cc of blood is drawn, in other embodiments, 27-110 cc of blood is drawn. Larger or smaller quantities of blood or plasma may be used as the starting material to provide proportionally larger or smaller quantities of the PRP composition. The blood or plasma may be from a single source or pooled from more than one source. In some embodiments, the PRP composition is isolated from the patient's own blood. In other embodiments, the PRP composition is derived from one or more histocompatible sources. In several embodiments, the source of blood or plasma may be allogenic, or pooled sources of platelets and/or plasma. In some embodiments, the PRP composition is neutrophil-depleted.

In some embodiments, the kit comprises one or more disposable separation tubes, such as GPS® II and GPS® Mini disposable separation tube from Cell Factor Technologies, Inc., that are adapted to be placed in a centrifugation device for concentrating platelets. In some embodiments, the kit may include a centrifuge for concentrating the platelets and white blood cells and optionally a device for neutrophil depletion such as taught in U.S. Pat. No. 7,462,268, which is incorporated herein by reference.

In some embodiments, the pH of the PRP composition is adjusted to physiological pH and the kit comprises a ph adjusting agent. The pH adjusting agent may be a biocompatible buffer such as HEPES, TRIS, monobasic phosphate, monobasic bicarbonate, or biocompatible buffer capable of adjusting the PRP composition to physiological pH. In some embodiments, the pH is adjusted to between 6.5 and 8.0. In some embodiments, the pH is adjusted to about 7.4. In some embodiments, the kit includes an anticoagulant such as citrate dextrose solution.

In some embodiments, the kit may comprise components to apply the PRP composition to a patient for treatment. The kit may include a sterilizing solution for treatment of the skin prior to administration of the PRP composition such as iodine (betadine). The kit may also include bandaging material to stop any bleeding caused by the withdrawal of blood or injection of PRP into the patient such as gauze pads and/or bandaid(s). In some embodiments, the kit includes a local anesthetic such as anbesol, benzocaine, lidocaine, procaine, bupivicaine, or any appropriate anesthetic known in the art. In some embodiments, the kit comprises syringes of appropriate size for preparation and administration of the PRP composition, and optionally, administration of the anesthetic. In several embodiments, the kit includes a procedure instruction sheet.

As used herein, the term “patient” refers to the recipient of a therapeutic treatment and includes all organisms within the kingdom animalia. In preferred embodiments, the animal is within the family of mammals, such as humans, bovine, ovine, porcine, feline, buffalo, canine, goat, equine, donkey, deer, and primates. The most preferred animal is human.

As used herein, the terms “treat” “treating” and “treatment” include “prevent” “preventing” and “prevention” respectively.

As used herein the term “an effective amount” of an agent is the amount sufficient to treat, inhibit, or prevent ischemia and/or reperfusion injury associated with indications and conditions including, but not limited to, myocardial infarction, arteriosclerosis. stroke, septic shock, traumatic shock, and associated with surgical procedures such as vascular interventional procedures including angioplasty, surgery that involves restriction of blood supply to an organ or tissue, abdominal surgery, abdominoplasty, adenoidectomy, amputation, angioplasty, appendectomy, arthrodesis, arthroplasty, brain surgery, cesarean section, cholecystectomy, colon resection, colostomy, corneal transplantation, discectomy, endarterectomy, gastrectomy, grafting of skin or other tissues, heart transplantation, liver transplantation, heart surgery hemicorporectomy, hemorrhoidectomy, hepatectomy, hernia repair, hysterectomy, kidney transplantation, laminectomy, laryngectomy, lumpectomy, lung transplantation, mammoplasty, mastectomy, mastoidectomy, myotomy, nephrectomy, nissen fundoplication, oophorectomy, orchidectomy, orthopedic surgery, parathyroidectomy, penectomy, phalloplasty, pneumonectomy, prostatectomy, radiosurgery, rotationplasty, splenectomy, stapedectomy, thoracotomy, thrombectomy, thymectomy, thyroidectomy, tonsillectomy, ulnar collateral ligament reconstruction, vaginectomy, vasectomy and any surgery involving cardiac bypass, cardiac artery bypass graft surgery and organ transplantation.

In addition to the foregoing uses for the compositions, methods and systems described herein, it will be apparent to those skilled in the art that other injured tissues, in addition to injured cardiac tissue and connective tissue, would benefit from the delivery of structural support materials to treat the injuries. Non-limiting examples of such tissues include the stomach, to reduce food intake and increase satiety; the abdominal wall, to prevent and treat hernias; and the bladder to prevent or treat incontinence. Such tissues may additionally include vascular tissues.

The following examples are provided for illustrative purposes only, and are in no way intended to limit the scope of the present embodiments.

### EXAMPLES

### Example 1

### Preparation of Platelet and WBC Enriched PRP

A platelet and WBC enriched (RevaCor) PRP composition was prepared from whole blood from the same human donor using a proprietary separation device (ThermoGenesis, Rancho Cordova, Calif.). After preparation, PRP was buffered to physiologic pH using 8.4% sodium bicarbonate and delivered to the myocardium without exogenous activation. The platelet and white blood cells counts were calculated for each trial before and after the PRP was prepared.

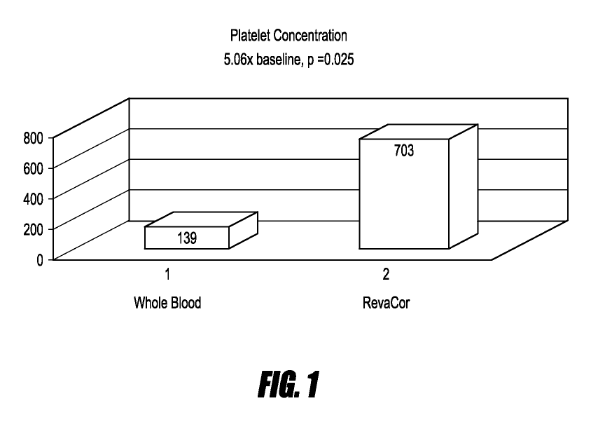

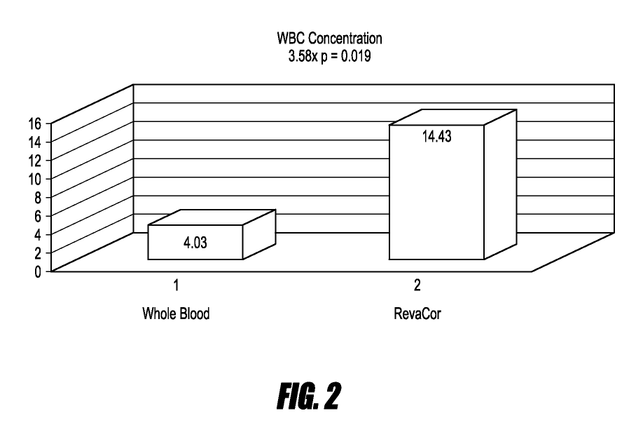

RevaCor PRP contained an average concentration of platelets 5.06 times baseline (p=0.025) and average concentration of white blood cells 3.6 times baseline (p=0.019). See FIGS. 1 and 2.

### Example 2

### Intramyocardial Administration of RevaCor PRP Improves LVEF and Reduces Scar Tissue Following Myocardial Ischemia-Reperfusion Injury

Six- to eight-week-old female NOD.Cg-Prkde^{scid} Il2rg^{tm1Wjl}/SzJ mice (Jackson Laboratory, Bar Harbor, Me.) were housed at the Stanford University animal care facility under standard temperature, humidity, and timed-lighting conditions and provided mouse chow and water ad libitum. All animals were handled in compliance with the National Research Council's guidelines for the care and use of laboratory animals.

Nineteen mice were selected and randomly assigned to experimental (RevaCor PRP-treated, n=10) or control (phosphate-buffered saline (PBS)-treated, n=9) groups. The mice were anesthetized and maintained with 3% isoflurane, intubated, and placed on a rodent ventilator. A left lateral thoracotomy was performed and the left anterior descending artery (LAD) was occluded with 8-0 Ethilon suture. The presence of ischemic myocardium confirmed adequate occlusion. After an occlusion time of 45 min, reperfusion of the LAD was allowed for 15 min. Following reperfusion, the mice were injected intramyocardially in the ischemic region via 23-gauge needle with either 50 μL of RevaCor PRP prepared as described in Example 1 (experimental group) or 50 μL PBS (controls). On post-operative day (POD) 21, mice were anesthetized with 1-3% isoflurane and underwent cardiac magnetic resonance imaging (MRI) using a small animal scanner to calculate left ventricle ejection fraction (LVEF) from the resulting video images.

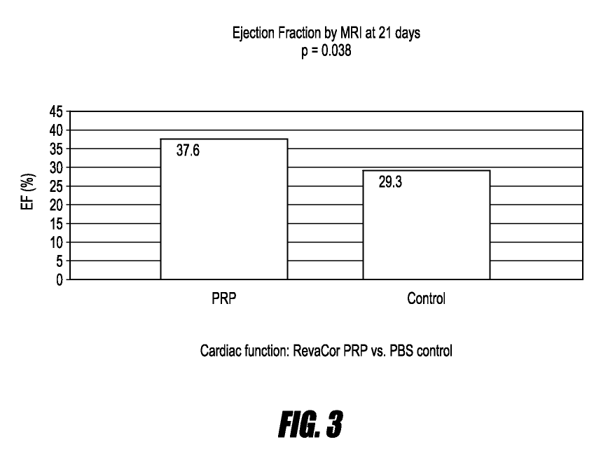

Compared with PBS controls, RevaCor PRP-treated animals showed a 28% improvement in LVEF compared 21 d post-operatively. Mean LVEF was 37.6±4.8% in the PRP-treated group (n=10) versus 29.3±9.7% in the PBS-treated group (n=9) with a P value=0.038. See FIG. 3.

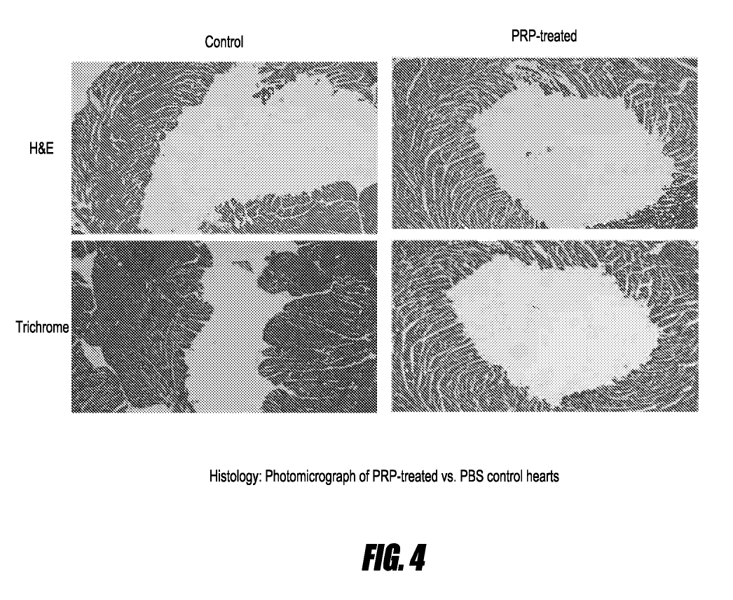

Following imaging and LVEF calculation, animals were euthanized on POD 21 and hearts harvested and processed for histology to examine myocardial fibrosis. Histologic analysis included hematoxylin, eosin, and trichrome staining. The presence of more scar tissue in the control group compared to the PRP group was detected with trichrome staining. See FIG. 4.

### Example 3

### Decreased Apoptosis of Hypoxic Human Microvascular Endothelial Cells Treated with PRP In Vitro

Human microvascular endothelial cells (HMEC-1) were maintained per manufacturer's instructions and cultured in supplemented D-MEM media (Cellgro, Manassas, Va.) on cell plates to 75-85% cellular confluence. The HMEC-1 plates were then incubated with and without 2.5% and 10% RevaCor PRP and placed in a humidified modular incubation chamber (model MIC-101, Billups-Rothenberg, Del Mar, Calif.), charged with a mixture of 1% O₂, 4% CO₂, and 95% N₂ to create hypoxic conditions and fully sealed before placement in a cell culture incubator at 37° C.

The plates were incubated at hypoxic conditions for periods of 6, 12, and 24 h. At each time point, HMEC-1 were collected, lysed with RIPA buffer containing a 10-fold dilution of protease inhibitor cocktail (Sigma-Aldrich, St. Louis, Mo.) and the cellular suspension was washed with PBS and the supernatants isolated and stored for analysis. The protein concentration of the supernatants was determined by bicinchoninic acid (BCA) assay (Promega, Madison, Wis.).

The supernatants were extracted with 4× NuPAGE sample buffer containing reducing agent (Invitrogen, Carlsbad, Calif.) for 5 min at 95° C., resolved by SDS-polyacrylamide gel electrophoresis (4-12% Tris-Bis NuPAGE mini gel), and transferred to nitrocellulose 0.2 μm pore membranes. Membranes were blocked with 5% milk/TBST for 30 min. at room temperature, incubated with the appropriate primary antibody at 4° C. overnight, and washed with TBST containing 0.05% Tween. Primary antibodies used were: actin (Abcam, Cambridge, Mass.); Bcl-2 (Santa Cruz Biotechnology, Santa Cruz, Calif.); caspase-9, cleaved caspase-3, cleaved PARP, free Bax, and GAPDH (all from Cell Signaling, Danvers, Mass.).

The appropriate HRP-conjugated secondary antibody, diluted in 5% milk/0.05% Tween/TBS was applied for 1 h at room temperature. The membranes were washed with 0.05% Tween/TBS and coated with ECL reagent (GE Healthcare, Chalfont St. Giles, United Kingdom) followed by signal detection with Hyperfilm ECL (GE Healthcare, Piscataway, N.J.). When necessary, membranes were stripped with Restore Stripping Buffer (Pierce, Rockford, Ill.), washed with water, and reprobed with appropriate primary antibody. Immunosignals were quantified using ImageJ software (National Institutes of Health, Bethesda, Md.).

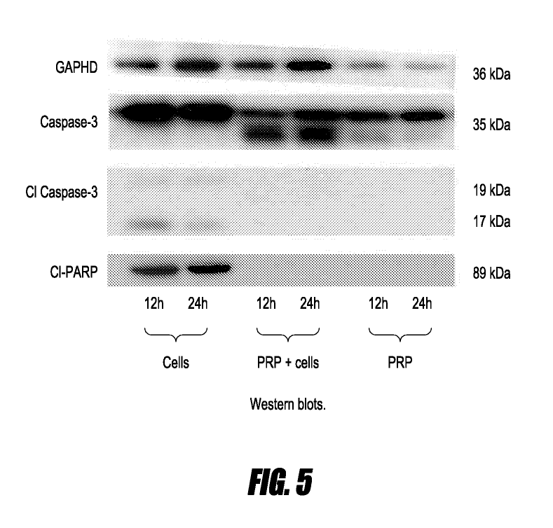

A significant decrease in the apoptosis markers, caspase-3 (Casp-3), cleaved caspase-3 (Cl-Casp-3), and cleaved PARP (Cl-PARP) were observed by Western blot analysis in hypoxic cells treated with PRP. There was also a trend towards decreased free Bax and increased Bcl-2 in PRP-treated groups compared to untreated hypoxic cells. Results are shown at FIG. 5 for cells exposed to hypoxia alone, for cells exposed to hypoxia plus PRP, and for PRP alone.

### Example 4

### Neutrophil-Depleted PRP Improves Cardiac Function Following Myocardial Injury

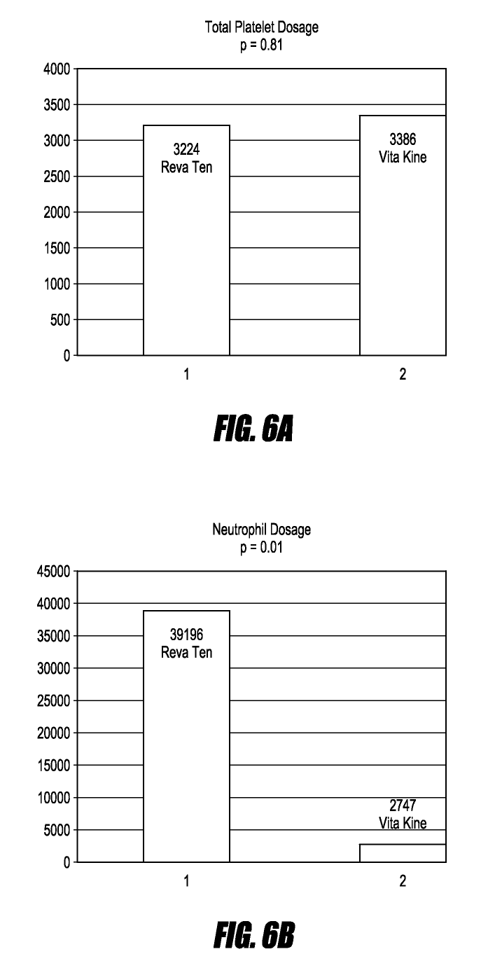

Two formulations of PRP, unfractionated PRP, which contains concentrated platelets and concentrated unfractionated WBCs and neutraphil-depleted PRP, which contains concentrated platelets and concentrated neutraphil-depleted WBCs were prepared as described herein using 60 ml of whole blood taken from each test subject prior to myocardial injury. The ratio of lymphocytes and monocytes to neutrophils is significantly higher in the neutraphil-depleted PRP compared to unfractionated PRP, while there is no significant difference in platelet dosages between the PRP compositions. See FIGS. 6A and 6B.

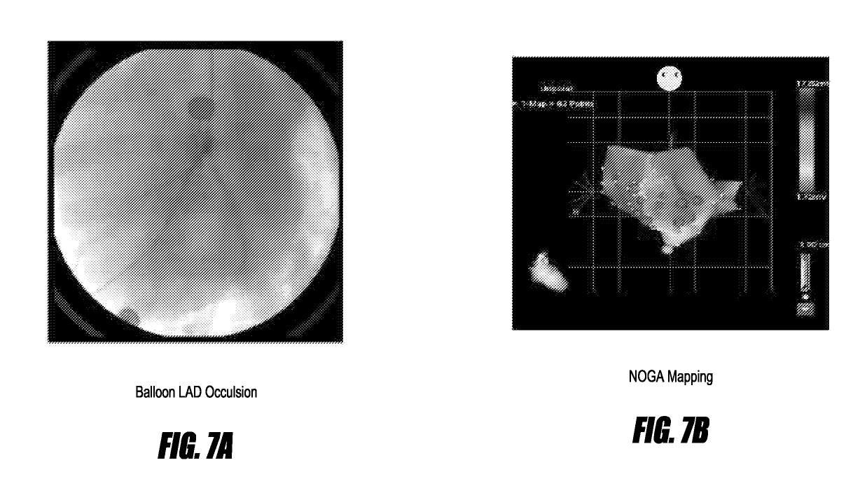

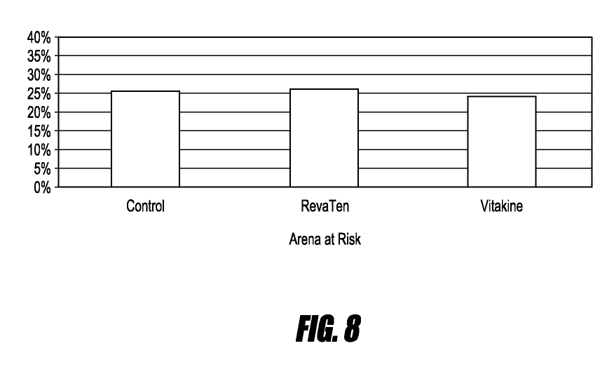

Twenty-two Yorkshire swine in three cohorts (control n=9, unfractionated PRP n=7, neutraphil-depleted PRP n=6) were subjected to myocardial ischemia-reperfusion injury by occluding the left anterior descending artery after the first septal branch with a balloon for sixty minutes prior to reperfusion. See FIG. 7A. NOGA electromechanical mapping was performed to identify regions of ischemic or malfunctioning myocardium. See FIG. 7B. Pathologic analysis did not demonstrate significant differences in area at risk for myocardial injury between the cohorts. See FIG. 8. Following NOGA mapping, animals in either the unfractionated PRP or neutraphil-depleted PRP cohorts received ten endomyocardial injections of either unfractionated PRP or neutraphil-depleted PRP in the region of myocardial injury defined by voltage criteria. Injections were performed with a Myostar injection catheter.

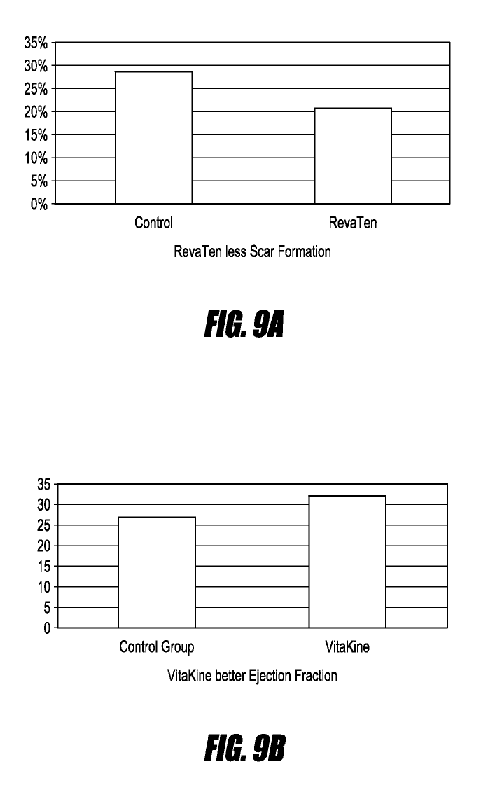

At 21 days post-injury, Cardiac Magnetic Resonance Imaging (Signa 3.0 T Excite HD Scanner; GE Health Systems, Milwaukee Wis.) was performed to calculate cardiac chamber size, ejection fraction, and scar tissue using gadolinium late enhancement. Treatment with unfractionated PRP did not demonstrate improvement in LVEF compared to control (28.1±11.5% vs. 27.1±4.7%; p=0.83), but did reveal a significantly smaller infarct size (21.4±7.1% vs. 28.9±6.7%; p=0.05 FIG. 9A). Treatment with neutraphil-depleted PRP demonstrated significant improvement in LVEF compared to control (33.3±6.0 vs. 27.1±4.7; p=0.04 See FIG. 9B), but did not reduce infarct size (27.5±5.2% vs. 28.9±6.7%, P=0.66).

### Example 5

### Treatment of Ischemic Heart Disease with Neutrophil-Depleted PRP

A human patient presents with symptoms of ischemic heart disease such as chest pain. The diagnostic evaluation including a physical exam, EKG, as well as laboratory studies determines that the patient has ischemic heart disease. A blood sample is drawn to create neutrophil-depleted PRP. The patient is taken to the catheterization laboratory to perform reperfusion therapy and then have neutrophil-depleted PRP applied, injected, or instilled. In another embodiment, the patient would go to the catheterization laboratory to have neutrophil-depleted PRP either injected or instilled in a delayed manner.

The neutrophil-depleted PRP in the above example can be prepared using a variety of techniques including, but not limited to, centrifuges, gravity filtration devices, cell sorting, or others. It can be combined with stem cells, genetic engineering or mechanical devices such as permanent or bioabsorbable pacemaker or stent. The neutrophil-depleted PRP can be autologous or made from allogenic sources. It can be made and then stored in a frozen or lyophilized state to be applied to the tissue later. In a preferred form it would be buffered to physiologic pH but it may also be valuable to instill neutrophil-depleted PRP at either acidic or basic pH for specific clinical indications such as ablation of an abnormal conduction pathway. In yet another embodiment, the neutrophil-depleted PRP could be prepared in a form that is depleted of other fractions of white blood cells, such as lymphocytes or monocytes, either partially or completely.

### Example 6

### Treatment of Acute Myocardial Infarction with Neutrophil-Depleted PRP

A human patient presents with symptoms of acute myocardial infarction such as chest pain. The diagnostic evaluation including a physical exam, EKG, as well as laboratory studies determines that the patient is having acute coronary syndrome such as unstable angina, Non-ST elevation myocardial infarction, or ST elevation myocardial infarction. A blood sample is drawn to create neutrophil-depleted PRP. The patient is taken to the catheterization laboratory to perform reperfusion therapy and then have neutrophil-depleted PRP applied, injected, or instilled to improve cardiac rhythm or protect against reperfusion arrhythmia. In another embodiment, the patient would go to the catheterization laboratory to have neutrophil-depleted PRP either injected or instilled in a delayed manner to prevent future arrhythmia.

The neutrophil-depleted PRP in the above example can be prepared using a variety of techniques including, but not limited to, centrifuges, gravity filtration devices, cell sorting, or others. It can be combined with stem cells, genetic engineering or mechanical devices such as permanent or bioabsorbable pacemaker or stent. The neutrophil-depleted PRP can be autologous or made from allogenic sources. It can be made and then stored in a frozen or lyophilized state to be applied to the tissue later. In a preferred form it would be buffered to physiologic pH but it may also be valuable to instill neutrophil-depleted PRP at either acidic or basic pH for specific clinical indications such as ablation of an abnormal conduction pathway. In yet another embodiment, the neutrophil-depleted PRP could be prepared in a form that is depleted of other fractions of white blood cells, such as lymphocytes or monocytes, either partially or completely.

### Example 7

### Treatment of Acute Myocardial Infarction with Neutrophil-Depleted PRP and Cardiomyocytes

A human patient presents with symptoms of acute myocardial infarction such as chest pain. The diagnostic evaluation including a physical exam, EKG, as well as laboratory studies determines that the patient is having acute coronary syndrome such as unstable angina, Non-ST elevation myocardial infarction, or ST elevation myocardial infarction. A blood sample is drawn to create neutrophil-depleted PRP. The patient is taken to the catheterization laboratory to perform reperfusion therapy and then have neutrophil-depleted PRP and cardiomyocytes applied, injected, or instilled. In some embodiments, the neutrophil-depleted PRP and cardiomyocytes are applied, injected, or instilled in conjunction with a Marlex mesh left ventricle restraint.

The neutrophil-depleted PRP in the above example can be prepared using a variety of techniques including, but not limited to, centrifuges, gravity filtration devices, cell sorting, or others. It can be combined with stem cells, genetic engineering or mechanical devices such as permanent or bioabsorbable pacemaker or stent. The neutrophil-depleted PRP can be autologous or made from allogenic sources. It can be made and then stored in a frozen or lyophilized state to be applied to the tissue later. In a preferred form it would be buffered to physiologic pH but it may also be valuable to instill neutrophil-depleted PRP at either acidic or basic pH for specific clinical indications such as ablation of an abnormal conduction pathway. In yet another embodiment, the neutrophil-depleted PRP could be prepared in a form that is depleted of other fractions of white blood cells, such as lymphocytes or monocytes, either partially or completely.

### Example 8

### Treatment of Peripheral Vascular Disease with Neutrophil-Depleted PRP Composition

A patient presents with symptoms of limb ischemia, which may include pain, limited walking distance and frank ulcerations on the leg. The clinician performing a history and physical examination notes decreased blood flow to the limb. Diagnosis of decreased blood flow can be confirmed with Ankle-Brachial indexes (ABIs) or other non-invasive technique, such as ultrasound including Doppler evaluations. The area of ischemia is identified. Neutrophil depleted PRP is injected into the ischemic area via a syringe, catheter or other delivery device. Specially designed catheters may be used to guide the clinician to the proper spot via an endovascular approach and then the PRP is delivered. Alternatively, the PRP can be directed injected into the muscle or surrounding tissue in the ischemic zone of the limb. Confirmation of delivery may be done via x-ray, ultrasound or other guidance tools. Measurement of success may be done via repeat physical examinations, ultrasound and or other imaging tools. Improvement in functional status such as walking distance or decrease rest pain can be expected. A kit specific for the use of neutrophil depleted PRP for peripheral vascular disease may be used that contains the appropriate materials. Measurement of VO2 max may also be done to evaluate patient status.

While methods, devices, and kits have been described in some detail here by way of illustration and example, such illustration and example may be for purposes of clarity of understanding only. It will be readily apparent to those of ordinary skill in the art in light of the teachings herein that certain changes and modifications may be made thereto without departing from the spirit and scope of the appended claims.

1. A formulation of platelet rich plasma that contains 1.01 times baseline platelets or more in combination with 1.01 times baseline white blood cells or more.

2. The formulation of claim 1, wherein monocytes and/or lymphocytes are increased in comparison to neutrophils.

3. The formulation of claim 1, wherein neutrophils are depleted to 1% or more of baseline levels.

4. A method of treating heart disease, lung disease, peripheral vascular disease, muscle, tendon, ligament, cartilage, bone, kidney, brain tissue and any other organ including pancreas, liver and skin comprising administering the formulation of claim 1 to an individual in need thereof.

5. A method of treating heart attack, congestive heart failure, chronic angina, critical limb ischemia, and/or any lung disease comprising administering the formulation of claim 1 to an individual in need thereof.

6. A method of measuring the value of monocytes and/or lymphocytes to neutrophils which comprises measuring the ratio of monocytes and/or lymphocytes to neutrophils.

7. The method of claim 6, wherein an increased ratio of monocytes and/or lymphocytes to neutrophils indicates that a PRP composition is suitable for use in treating heart attack, congestive heart failure, chronic angina, critical limb ischemia and/or any lung disease.

8. A composition comprising:
platelets derived from whole blood at a first concentration of at least about 1.1 times a platelet concentration in the whole blood;
white blood cells derived from the whole blood at a second concentration of at least a white blood cell concentration in the whole blood, wherein the white blood cells comprise:
neutrophils at a third concentration, wherein the third concentration is less than the neutrophil concentration in the whole blood;
lymphocytes at a fourth concentration of at least 1.1 times a lymphocyte concentration in the whole blood; and
monocytes at a fifth concentration of about 1.1 times a monocyte concentration in the whole blood.

9. The composition of claim 8, wherein the third concentration is 1% of the concentration of neutrophils in whole blood

10. The composition of claim 8, wherein the neutrophils are substantially eliminated.

11. The composition of claim 8, wherein the third concentration is between about 2,000 neutrophils per microliter and about 3,000 neutrophils per microliter.

12. A method of treating a cardiac condition comprising:
identifying cardiac ischemia in a patient selected from the group consisting of ischemic heart disease or myocardial infarction; and
delivering the composition of claim 8 to the patient to treat the cardiac condition.

13. The method of claim 12, further comprising preparing the composition from the whole blood of the patient.

14. The method of claim 12, further comprising testing a platelet rich plasma for conformance to the composition prior to delivering the composition.

15. The method of claim 12, wherein acute ischemic tissue damage has occurred within the last 24 hours.

16. The method of claim 12, wherein reperfusion therapy to treat a myocardial infarction has occurred within the last 24 hours and wherein delivering the composition occurs within about 24 hours before or after the reperfusion therapy.

17. The method of claim 12, wherein the composition further comprises one or more of fetal cardiomyocytes, embryonic stem cells, bone marrow cells, induced pluripotent stem cells, and cardiomyocytes derived from induced pluripotent stem cells.

18. The method of claim 12, wherein the composition further comprises a biomaterial scaffold comprised of one or more of gelatin, alginate, collagen type 1 and Matrigel, polyglycolide, collagen, fibrin, or self-assembling peptides.

19. A method of reducing apoptosis in ischemia damaged tissue, comprising:
delivering a PRP composition to a site of ischemic damage.

20. The method of claim 19, wherein the PRP composition comprises:
platelets derived from whole blood at a first concentration of at least about 1.1 times a platelet concentration in the whole blood;
white blood cells derived from the whole blood at a second concentration of at least about 1.1 times a white blood cell concentration in the whole blood.

21. The method of claim 20, wherein the white blood cells comprise:
neutrophils, wherein the neutrophil concentration is less than the neutrophil concentration in the whole blood;
lymphocytes, wherein the lymphocyte concentration is 1.1 times lymphocyte concentration in the whole blood; and
monocytes, wherein the monocyte concentration is 1.1 times monocyte concentration in the whole blood.

22. A kit comprising:
a separating device for separation of whole blood into components for preparation of a platelet-containing composition,
one or more collection devices,
one or more means for sterilization,
needle or catheter sufficient for injection of the platelet-containing composition.

23. The kit of claim 22 wherein the separation device provides a blood product enriched in platelets and depleted in neutrophils.

24. The kit of claim 23, wherein the neutrophils are depleted to an amount of less than 80%, preferably less than 90%, more preferably less than 95% and more preferably less than 99% the levels found in whole blood.

25. The kit of claim 22, further comprising a measurement device to test the efficacy of the platelet-containing composition for treatment of a pre-determined disease.

26. The kit of claim 25, wherein the disease is cardiovascular disease, lung disease, peripheral vascular disease, muscle, tendon, ligament, cartilage, bone, kidney, brain tissue and any other organ including pancreas, liver, a limb and skin.

27. The kit of claim 22, wherein the collection devices comprise one or more syringes, apheresis needles or other device for collecting blood from a patient.

28. The kit of claim 22 further comprising a pH adjusting agent.

29. A method of treating a peripheral vascular disease comprising:
identifying ischemia in a limb of a patient; and
delivering the composition of claim 8 to the patient to treat the ischemia.

30. The method of claim 29, further comprising preparing the composition from the whole blood of the patient.

31. The method of claim 29, further comprising testing a platelet rich plasma for conformance to the composition prior to delivering the composition.
